(19) United States
(12) Patent Application Publication
(10) Pub. No.: US 2012/0101000 A1
(43) Pub. Date: Apr. 26, 2012

(54) HIGH COMPLEXITY MAMMALIAN DISPLAY LIBRARY AND METHODS OF SCREENING

(76) Inventor: Chen Zhou, San Jose, CA (US)

(21) Appl. No.: 13/130,561

(22) PCT Filed: Nov. 20, 2009
(86) PCT No.: PCT/US09/65393

§ 371 Date: Aug. 3, 2011

(30) Foreign Application Priority Data
Nov. 21, 2008 (US) 61/117028
Sep. 18, 2009 (US) 61/276956

(51) Int. Cl. C40B 30/04; C40B 40/02; C12N 15/85; C40B 50/00

(52) U.S. Cl. 506/9; 506/23; 506/14; 435/320.1

(57) ABSTRACT

Provided herein are methods of isolating a polynucleotide encoding a polypeptide such as an antibody with a desired property by way of mammalian display library screening and methods of generating a library of polynucleotides encoding polypeptides such as antibodies, wherein the polynucleotides collectively encode at least 109 different polypeptides. Also provided are kits for carry out the methods described herein, polynucleotides isolated by methods described herein, libraries encoding the antibody reservoir of different species including human, mouse, rabbit, and polypeptides encoded by the polynucleotides.

### RELATED APPLICATIONS

This application claims priority benefit to U.S. Provisional Patent Application No. 61/117,028, filed on Nov. 21, 2008 and U.S. Provisional Patent Application No. 61/276,956, filed on Sep. 18, 2009, the content of each of which is incorporated by reference in their entireties.

### TECHNICAL FIELD

This application pertains to the construction and screening of mammalian polypeptide display libraries, particularly mammalian display libraries of full length antibodies.

### BACKGROUND

High throughput screening for antibodies that bind specifically to a target antigen was made possible by cell surface display technologies, including phage display, ribosomal/mRNA display, and yeast display. Hoogenboom et al., Nature Biotechnology (2005), 23(9):1105-1116). While each of these screening platforms has its specific advantages, they are all based on expression of antibodies in an environment that is different from that of mammalian cells. Protein folding, glycosylation, disulfide bond formation, and/or modification are likely different from proteins produced in those systems versus in mammalian cells.

To circumvent these problems, selection platforms based on the expression of antibodies in their natural environment, i.e., mammalian cells, have been developed. For example, researches have developed antibody display technologies that allow small libraries of immunoglobulins be displayed on the surface of mammalian cells. See, e.g., Higuchi et al., J. Immun. Methods (1997) 202:193-204. One major drawback of performing antibody screening in mammalian cells is the limited number of antibodies that can be screened. Because each mammalian cell can take up a large number of different polynucleotides encoding different antibodies during transfection, isolation of an antibody-encoding polynucleotide from an antibody-producing mammalian cell is difficult and tedious. Thus, efforts in the area of mammalian display have been focusing on establishing mammalian display systems in which a smaller number of different polynucleotides are present in each mammalian cell. For example, WO05/063817 discloses a method of screening a small library of full length antibodies. After homologous integration of a single-gene copy in each cell, the population was sorted by flow cytometry to obtain cells expressing antibodies with high binding affinity. See also US2005/0059082 and WO08/070,367. Most of the currently available mammalian display technologies are infeasible for screening a high complex library of polynucleotides encoding a large number of different antibodies. While phage display technology allows for the screening of 10¹⁰ to even 10¹⁵ clones in a single panning round, the throughput of a mammalian display screening procedure in currently available methods is limited to the concomitant analysis of about 10⁶ to 10⁷ clones. There is thus a need for screening methods and systems that allow high complexity mammalian display library screening.

The disclosures of all publications, patents, patent applications and published patent applications referred to herein are hereby incorporated herein by reference in their entirety.

### BRIEF SUMMARY OF THE INVENTION

The present invention provides methods of screening mammalian display libraries, including methods of screening for polynucleotides encoding a polypeptide having a desired property, methods of enriching polynucleotides encoding a polypeptide having a desired property, methods of isolating a mammalian cell containing a polynucleotide encoding a polypeptide having a desired property, methods of screening for mammalian cells containing a polynucleotide encoding a polypeptide having a desired property, methods of identifying a polypeptide having a desired property, and methods of obtaining a polypeptide having a desired property.

In some embodiments, the method comprises: a) screening an initial population of mammalian cells transfected (such as transiently transfected) with a primary library of polynucleotides for cells displaying a polypeptide having the desired property and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying a polypeptide having the desired property and recovering a second subpopulation of mammalian cells. In some embodiments, steps b) and c) are repeated more than once. In some embodiments, the method comprises: a) reverse transcribing mRNA extracted from a subpopulation of mammalian cells into cDNA and amplifying said cDNA to generate a sublibrary of polynucleotides, wherein the subpopulation of cells is obtained by screening an initial population of mammalian cells transfected (such as transiently transfected) with a primary library of polynucleotides for cells displaying a polypeptide having the desired property; b) screening a population of mammalian cells transfected with the sublibrary of polynucleotides for cells displaying a polypeptide having the desired property and recovering a second subpopulation of mammalian cells. In some embodiments, the method comprises screening a population of mammalian cells transfected with a sublibrary of polynucleotides for cells displaying a polypeptide having the desired property and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying a polypeptide having the desired property. In some embodiments, the polypeptide is a heteromeric protein. In some embodiments, the polypeptide is an antibody.

In some embodiments, there is provided a method of isolating a polynucleotide encoding a polypeptide having a desired property, comprising: a) screening an initial population of mammalian cells transfected (such as transiently transfected) with a primary library of polynucleotides for cells displaying a polypeptide having the desired property and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying a polypeptide having the desired property and recovering a second subpopulation of mammalian cells; and d) isolating from said second subpopulation of mammalian cells a polynucleotide encoding a polypeptide having the desired property.

In some embodiments, the polynucleotides in the primary library encode at least 10⁹ (including for example about any of 10¹⁰, 10¹¹, 10¹², 10¹³, 10¹⁴, 10¹⁵, and 10¹⁶) different polypeptides. In some embodiments, the initial population of mammalian cells is transiently transfected with the primary library under a condition where an individual mammalian cell in the population can take up more than about 100, such as more than about any of 10³, 10⁴, 10⁵, or 10⁶ copies of different polynucleotides. In some embodiments, the screening condition in step c) is more stringent than that of step a).

In some embodiments, therein is provided a method of isolating a polynucleotide that encode a heteromeric protein with a desired property, comprising: a) screening an initial population of mammalian cells for cells displaying a heteromeric protein having a desired property and recovering a subpopulation of mammalian cells, wherein the initial population of mammalian cells are transfected (such as transiently transfected) with a primary library of polynucleotides encoding a first polypeptide and a second polypeptide and wherein the first polypeptide and the second polypeptide form a heteromeric protein on the cell surface; b) reverse transcribing mRNA extracted from said subpopulation of mammalian cells into cDNA and amplifying said cDNA to generate a sublibrary of polynucleotides; c) screening a subsequent population of mammalian cells for cells displaying a heteromeric protein with a desired property and recovering a second subpopulation of mammalian cells, wherein the subsequent population of mammalian cells are transfected with the sublibrary of polynucleotides encoding a first polypeptide and a second polypeptide and wherein the first polypeptide and the second polypeptide form a heteromeric protein; and d) isolating from said second subpopulation of mammalian cells polynucleotides encoding a polypeptide having the desired property.

In some embodiments, there is provided a method of isolating a polynucleotide encoding an antibody specifically recognizing an antigen, comprising: a) screening an initial population of mammalian cells transfected (such as transiently transfected) with a primary library of polynucleotides encoding antibodies for cells displaying an antibody specifically recognizing the antigen and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying an antibody specifically recognizing the antigen and recovering a second subpopulation of mammalian cells; and d) isolating from said second subpopulation of mammalian cells a polynucleotide encoding an antibody specifically recognizing the antigen. In some embodiments, the polynucleotides in the primary library encode at least 10⁹ different antibodies. In some embodiments, step a) comprises: (i) contacting the initial population of mammalian cells with the antigen under a suitable binding condition; and (ii) recovering a subpopulation of mammalian cells that bind to the antigen. In some embodiments, step c) comprises: (i) contacting the enriched mammalian display library with the antigen under a suitable binding condition; and (ii) recovering a subpopulation of mammalian cells that bind to the antigen. In some embodiments, step d) comprises: i) isolating mRNA from the subpopulation of cells, ii) amplifying the mRNA into cDNA; iii) cloning the cDNA into a cloning vector; and iv) determining the sequence of the DNA.

In some embodiments, there is provided a method of isolating a polynucleotide encoding an antibody specifically recognizing a specific antigen, comprising: a) screening an initial population of mammalian cells transiently transfected with a first primary library of polynucleotides encoding light chain and a second primary library encoding heavy chain and displaying antibodies encoded by the polynucleotides on the cell surface to obtain a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides and displaying antibodies encoded by the polynucleotides on the cell surface to obtain a second subpopulation of mammalian cells; and d) isolating from said second subpopulation of mammalian cells a polynucleotide encoding an antibody specifically recognizing a specific antigen. In some embodiments, steps b) and c) are repeated at least once prior to step d). In some embodiments, steps b) and c) are repeated no more than about three times prior to step d). In some embodiments, the polynucleotides in the primary libraries encode at least 10⁹ different antibodies. In some embodiments, step a) comprises: (i) contacting the initial population of mammalian cells with the antigen under a suitable binding condition; and (ii) recovering a subpopulation of mammalian cells that bind to the antigen. In some embodiments, step c) comprises: (i) contacting the enriched mammalian display library with the antigen under a suitable binding condition; and (ii) recovering a subpopulation of mammalian cells that bind to the antigen. In some embodiments, step d) comprises: i) isolating mRNA from the subpopulation of cells, ii) amplifying the mRNA into cDNA; iii) cloning the cDNA into a cloning vector; and iv) determining the sequence of the DNA. In some embodiments, the initial population of mammalian cells is transiently transfected with the primary libraries under a condition where individual mammalian cells in the population can take up more than about 100, such as more than about any of 10³, 10⁴, 10⁵, or 10⁶ copies of different polynucleotides. In some embodiments, the method further comprises transiently transfecting the primary libraries of polynucleotides into the initial population of mammalian cells.

In some embodiments, there is provided a method of isolating a polynucleotide that encodes an antibody specifically recognizing an antigen, comprising: a) screening an initial population of mammalian cells transfected with a first primary library of polynucleotides encoding a light chain and second primary library of polynucleotides encoding a heavy chain for cells displaying an antibody specifically recognizing the antigen and recovering a subpopulation of mammalian cells; b) reverse transcribing mRNA extracted from said subpopulation of mammalian cells into cDNA and amplifying said cDNA to generate a first sublibrary of polynucleotides encoding the light chain and a second sublibrary of polynucleotides encoding the heavy chain; c) screening a population of mammalian cells transfected with the first sublibrary of polynucleotides and the second sublibrary of the polynucleotides for cells displaying an antibody specifically recognizing the antigen and recovering a second subpopulation of mammalian cells; d) reverse transcribing mRNA extracted from said second subpopulation of mammalian cells into cDNA and amplifying said cDNA to generate a third sublibrary of polynucleotides encoding the light chain and the heavy chain; e) screening a population of mammalian cells transfected with the third sublibrary of polynucleotides for cells displaying an antibody specifically recognizing the antigen and recovering a third subpopulation of mammalian cells; and f) isolating from said third subpopulation of mammalian cells a polynucleotide encoding an antibody specifically recognizing the antigen.

In another aspect, there is provided a population of mammalian cells transiently transfected with polynucleotides encoding antibodies, wherein the polynucleotides collectively encode at least about 10⁹ different recombinant antibodies.

Also provided are libraries and kits for carrying out the methods described herein. Further provided are sublibraries generated during the screening process and polynucleotides/polypeptides obtained from methods described herein.

### BRIEF DESCRIPTION OF THE FIGURES

FIG. 1 provides a schematic diagram showing an exemplary method of screening as described herein.

FIG. 2 provides a schematic diagram showing an exemplary single cassette expression vector. Enzymes 1-3 refer to restriction enzymes cleavage sites recognizable by a different restriction enzyme (such as restriction enzymes that recognize a non-palendromic sequence), wherein the ends of each fragment resulting from the cleavage with said restriction enzyme do not self-ligate.

FIG. 3 provides a schematic illustration of the procedure to construct the single cassette expression vector pDGB-HC-TM. The HC-TM fragment was generated by RT-PCR amplification and PCR assembling. The restriction enzyme-recognizing sequences were incorporated into the fragment during PCR reaction. The fragment was inserted into the vector pcDNA5-FRT to form the interim vector. Then the hygromycin expression cassette was deleted from interim vector to form the vector pDGB-HC-TM.

FIG. 4 shows the results of restriction enzyme digestion of vector pDGBHC-TM. The vector was digested by 5 restriction enzymes in four ways and analyzed by electrophoresis in 1% agarose gel. M: 1 kb plus DNA ladder (Invitrogen); A: Nhel+Xhol; B: Sfil; C: BsmBI; D: BstXI.

FIG. 5A provides a schematic illustration of the procedure to construct the dual cassette expression vector. Four fragments were PCR-amplified and ligated together by 4-way ligation to form the vector pDGB4. Then FCS was inserted in the vector to form the vector pDGB4-FCS.

FIG. 5B shows the results of restriction enzyme digestion analysis of vector pDGB4. Purified plasmid DNA of pDGB4 clone #16 was digested by 3 restriction enzymes in 5 ways and electrophoresis analyzed in 1% agarose-TBE gel. M: DNA ladder; 1: SfiI I; 2: BstX I; 3: BsmB I; 4: BstX I+Sfi I; 5: BsmB I+Sfi I.

### DETAILED DESCRIPTION OF THE INVENTION

The present invention provides a high efficiency method of screening mammalian display library for polypeptides with a desired property. The method is based on the fact that, although each mammalian cell transfected with foreign DNA plasmids can contain up to 10⁶ copies of plasmids, only a small portion of (for example about 10²-10³ copies of) plasmids are transcribed into mRNA and expressed into polypeptides. Accordingly, the mRNA from positive cells obtained from a mammalian display library screen would be significantly less complex (for example at least 1000 times less complex) than that of the DNA in those cells. The method of the present invention takes advantage of this reduction in complexity.

After an initial screening of a primary mammalian display library for cells displaying polypeptides with a desired property and obtaining a subpopulation of mammalian cells that are enriched with those cells, mRNA from the enriched subpopulation of mammalian cells is isolated and amplified by methods such as RT-PCR (reverse transcriptase PCR) to create a pool of polynucleotides (a sublibrary). This pool of polynucleotides is in turn introduced into mammalian cells and subject to another round of library screening. The second round of screening process can be repeated, for example, once, twice, or more times. The method described herein allows high efficiency screening of mammalian display library, making it possible to screen mammalian display libraries with a diversity of at least 10⁹.

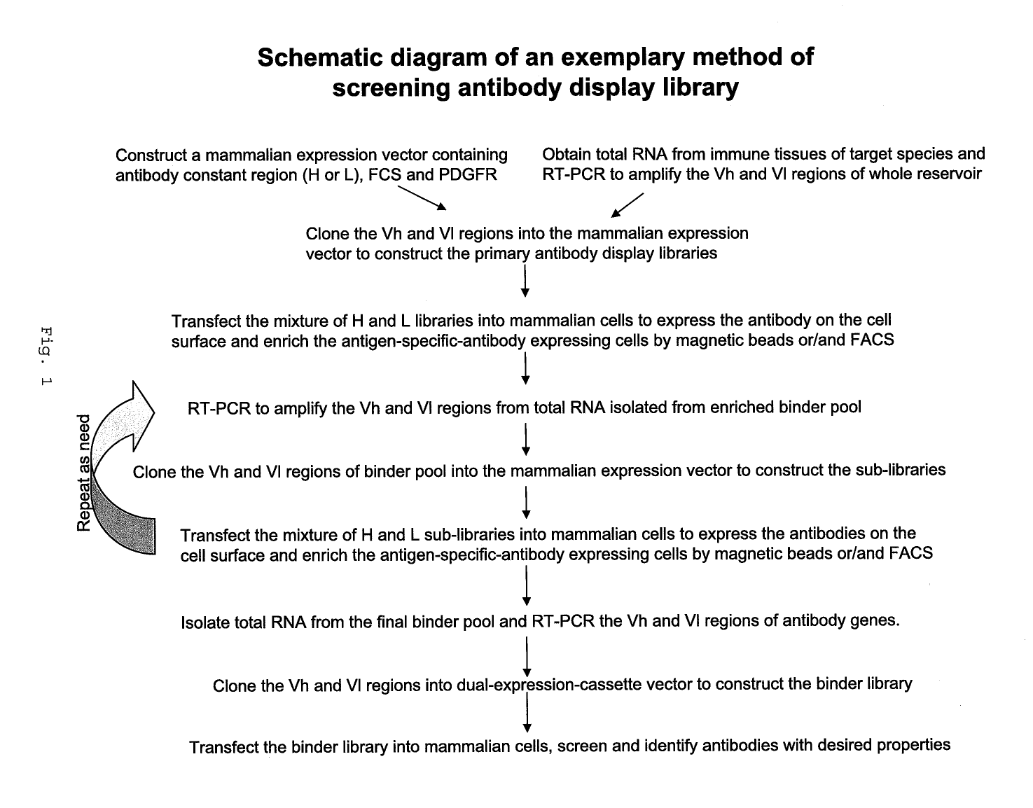

The methods provided herein are particularly suitable for screening heteromeric proteins. For example, methods of screening antibodies are provided. In one exemplary method, a primary library of antibody light chain and a primary library of antibody heavy chain are co-transfected into mammalian cells, allowing display of antibodies on the cell surface. After an initial screening of the mammalian cells to obtain a subpopulation of mammalian cells that is enriched with cells expressing the desired antibody, mRNA is extracted from the subpopulation of mammalian cells, amplified by RT-PCR, and cloned into suitable vectors to generate a sublibrary of heavy chain and a sublibrary of light chain. The sublibraries of heavy chain and light chain are in turm introduced into mammalian cells and subject to another round of library screening. The second round of screening can be repeated, for example, once, twice, or more times. During the final phase of the screening method, the antibody light and heavy chains are cloned into a dual expression cassette vector to generate a single antibody library (as opposed to two different libraries). The single library is then subject to a final round of screening to obtain polynucleotides that encode the desired antibody. FIG. 1 further provides a schematic diagram showing an exemplary method of screening antibody display libraries.

Accordingly, the invention in one aspect provides a method of screening a mammalian display library or method of isolating a polynucleotide encoding a polypeptide (such as an antibody) with a desired property by way of mammalian display library screening.

In another aspect, there is provided a population of mammalian cells transiently transfected with polynucleotides encoding polypeptides (such as antibodies), wherein the polynucleotides collectively encode at least 10⁹ different recombinant polypeptides on the mammalian cell surface.

In another aspect, there is provided a method of generating a library of polynucleotides for mammalian display screening, wherein the polynucleotides collectively encode at least 10⁹ different recombinant polypeptides (such as antibodies).

Also provided are kits for carry out the methods described herein. Also provided are polynucleotides isolated by methods described herein and polypeptides encoded by the polynucleotides.

It is to be understood that both the foregoing general description and the following detailed description are exemplary and explanatory only and are not restrictive of the compositions and methods described herein. In this application, the use of the singular includes the plural unless specifically stated otherwise. Also, the use of “or” means “and/or” unless state otherwise. Similarly, “comprise,” “comprises,” “comprising,” “include,” “includes” and “including” are not intended to be limiting.

It is understood that aspect and embodiments of the invention described herein include “consisting” and/or “consisting essentially of” aspects and embodiments.

### DEFINITIONS

As used herein, the following terms and phrases are intended to have the following meanings:

“Display” or “mammalian display” refers to presentation of different recombinant polypeptides on the surface of mammalian host cells.

“Library” used herein refers to a diverse collection or mixture of polynucleotides comprising polynucleotides encoding different recombinant polypeptides. In certain embodiments, a library of polynucleotides may comprise at least 10⁶, 10⁷, 10⁸, 10⁹, 10¹⁰, 10¹¹, 10¹², 10¹³, 10¹⁴, 10¹⁵, or more different polynucleotides within a given collection of polynucleotides. Typically, the different polynucleotides in the library are related through, for example, their origin from a single animal species (for example, human, mouse, rabbit, goat, horse), tissue type, organ, or cell type. A “library” may comprise polynucleotides of a common genus. For example, the genus can be polynucleotides encoding an immunoglobulin subunit polypeptide of a certain type and class e.g., a library might encode an antibody μ, γ1, γ2, γ3, γ4, a 1, a2, e, or d heavy chain, or an antibody κ or λ light chain. Although each member of any one library described herein may encode the same heavy or light chain constant region, the library may collectively comprise at least 10⁶, 10⁷, 10⁸, 10⁹, 10¹⁰, 10¹¹, 10¹², 10¹³, 10¹⁴, or, 10¹⁵ different variable regions i.e., a “plurality” of variable regions associated with the common constant region.

“Mammalian display library” refers to a library comprising polynucleotides encoding different recombinant polypeptides that can be displayed on mammalian cells. In some contexts, the term is also used generally to include mammalian cells transfected with the library of polynucleotides.

“Diversity” of a library refers to the number of different recombinant polypeptides encoded by the polynucleotides in the library.

“Screening” used herein refers to the method in which a pool comprising the desired species is subject to an assay in which the desired species can be detected, and subsequently an aliquot of the pool in which the desired species is detected and optionally enriched is recovered or obtained.

“Recovering” is used herein to mean a crude separation of a desired species from the rest of the pool which are not desired.

“Stringency” of a screening condition refers to the assay condition for the screening method. For example, when an assay for binding to a specific binding partner is carried out, stringency of the screening condition refers to the stringency of the condition for the binding assay.

By “a population of mammalian cells” is meant a group of mammalian cells into which a library of polynucleotides can be introduced and displayed. Although it is preferred that a population of cells be a monoculture, i.e., wherein each cell in the population is of the same cell type, mixed cultures of cells are also contemplated. Cells may be adherent, i.e., cells which grow attached to a solid substrate, or, alternatively, the cells may be in suspension. The mammalian cells may be cells derived from primary tumors, cells derived from metastatic tumors, primary cells, cells which have lost contact inhibition, transformed primary cells, immortalized primary cells, cells which may undergo apoptosis, and cell lines derived there from.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab', F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

“Humanized” antibodies refer to a molecule having an antigen-binding site that is substantially derived from an immunoglobulin from a non-human species and the remaining immunoglobulin structure of the molecule based upon the structure and/or sequence of a human immunoglobulin. The antigen-binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody, which are also termed one or more CDRs “derived from” one or more CDRs.

“Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non human host organisms in combination with constant regions derived from, for example, human cell preparations. While the variable region has the advantage of ease of preparation, and the specificity is not affected by its source, the constant region being human, is less likely to elicit an immune response from a human subject when the antibodies are injected than would the constant region from a non-human source. However, the definition is not limited to this particular example.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

An antibody or a polypeptide “specifically binds” or “preferentially binds” to an antigen or a target if it binds with greater affinity, avidity, more readily, and/or with greater duration than it binds to other substances. It is understood by reading this definition that, for example, an antibody or a polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The terms “full length antibody,” “intact antibody,” and “whole antibody” are used herein interchangeably, to refer to an antibody in its substantially intact form, not antibody fragments. The term particularly refers to an antibody with heavy chains that contains the Fc region. A full length antibody can be a native sequence antibody or an antibody variant.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single antibody; (vi) the dAb fragment which consists of a VH domain; (vii) isolated CDR regions; (viii) F(ab′).sub.2 fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) “linear antibodies” comprising a pair of tandem Fd segments (VH-CH1-VH-CH1) which, together with complementary light chain polypeptides, form a pair of antigen binding regions.

The term “heavy chain” as used herein refers to the larger immunoglobulin subunit which associates, through its amino terminal region, with the immunoglobulin light chain. The heavy chain comprises a variable domain and a constant domain. The constant domain further comprises the CH1, hinge, CH2, and CH3 domains. In the case of IgE, IgM, and IgY, the heavy chain comprises a CH4 domain but does not have a hinge domain. The phrase “immunoglobulin heavy chain constant domain” refers to the CH1, hinge, CH2, CH3, CH4 domains or any combination thereof.

The term “light chain” as used herein refers to the smaller immunoglobulin subunit which associates with the amino terminal region of a heavy chain. As with a heavy chain, a light chain comprises a variable region and a constant region. There are two different kinds of light chains, kappa and lambda, referred to herein as “immunoglobulin light chain constant domains.” A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

The term “antibody variant” as used herein refers to an antibody with single or multiple mutations in the heavy chains and/or light chains. In some embodiments, the mutations exist in the variable region. In some embodiments, the mutations exist in the constant region.

“Nucleic acid” or “polynucleotide” or grammatical equivalents as used herein means at least two nucleotides covalently linked together. Nucleic acids and polynucleotides are polymers of any length, including, e.g., 200, 300, 500, 1000, 2000, 3000, 5000, 7000, 10,000, etc. A nucleic acid described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring nucleic acids and analogs can be made; alternatively, mixtures of different nucleic acid analogs, and mixtures of naturally occurring nucleic acids and analogs may be made.

The terms “polypeptide” used herein refers to polymers of amino acid residues. The term “polypeptide” used herein encompasses protein, peptides, and heteromeric proteins. The term polypeptide also applies to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses a heteromeric protein, such as an antibody (for example a full length antibody).

“Heteromeric protein” used herein refers to a protein having at least two polypeptide chains, at least two of which are different from each other. For example, the heteromeric protein can be an antibody having a light chain and a heavy chain. In some embodiments, the heteromeric protein is a T cell receptor. In some embodiments, the heteromeric protein is an antibody-like peptibody as described herein.

“Peptibody” used herein refers to a chimeric molecule in which a polypeptide is fused to the N-terminus of a constant region of the heavy chain or light chain of an antibody. For example, the polypeptide can be fused to the Fc region of an antibody heavy chain. Alternatively, the polypeptide can be fused to a full length constant region of the heavy chain. The polypeptide can also be fused to the CL region of the antibody light chain.

By the term “recombinant” polynucleotide herein is meant a polynucleotide not normally found in its natural environment. It is understood that once a recombinant polynucleotide is made and reintroduced into a host cell or organism, it will replicate non-recombinantly, e.g., using the in vivo cellular machinery of the host cell rather than in vitro manipulations; however, such nucleic acids, once produced recombinantly, although subsequently replicated non-recombinantly, are still considered recombinant for the purposes of the invention.

The term “recombinant polypeptide” is a polypeptide made using recombinant techniques, e.g., through the expression of a recombinant nucleic acid as depicted above.

The term “heterogonous” when used with reference to portions of a nucleic acid indicates that the nucleic acid comprises two or more subsequences that are not normally found in the same relationship to each other. For instance, the nucleic acid is typically recombinantly produced, having two or more sequences, e.g., from unrelated genes arranged to make a new functional nucleic acid, e.g., a promoter from one source and a coding region from another source. Similarly, a heterogonous protein will often refer to two or more subsequences that are not found in the same relationship to each other, e.g., a fusion protein.

A “promoter” is typically an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is active under environmental or developmental regulation.

“Operably linked” refers to a juxtaposition of two or more components, wherein the components so described are in a relationship permitting them to function in their intended manner. For example, a promoter and/or enhancer is operably linked to a coding sequence if it acts in cis to control or modulate the transcription of the linked sequence. Generally, but not necessarily, the DNA sequences that are “operably linked” are contiguous and, where necessary to join two protein coding regions or in the case of a secretory leader, contiguous and in reading frame. However, although an operably linked promoter is generally located upstream of the coding sequence, it is not necessarily contiguous with it. A polyadenylation site is operably linked to a coding sequence if it is located at the downstream end of the coding sequence such that transcription proceeds through the coding sequence into the polyadenylation sequence. Linking is accomplished by recombinant methods known in the art, e.g., using PCR methodology, by annealing, or by ligation at convenient restriction sites. If convenient restriction sites do not exist, then synthetic oligonucleotide adaptors or linkers are used in accord with conventional practice.

The term “transfected” or “transformed” or “transduced” as used herein refers to a process by which exogenous nucleic acid is transferred or introduced into the host cell. A transformed cell includes the primary subject cell and its progeny. The host cell can be bacteria, yeast, mammalian cell, and plant cells.

A “cell surface tether domain” as used herein, refers to an amino acid sequence that confers the ability of a polypeptide to be associated with a host cell outer membrane, and which is sometimes but not always naturally present in the protein of interest. As described herein, cell surface tether domains include, for example, transmembrane domains or glycosidylphosphatidylinositol (GPI) signal sequences.

The term “expression vector” as used herein refers to a self-replicating polynucleotide and, in the present invention, comprises an expression construct. The expression vector will comprise at least one replication origin (also referred to as “origin of replication”). The replication origin confers the ability to replicate in a host and may be viral, eukaryotic, or prokaryotic. The expression vector may be used to stably or transiently transfect a eukaryotic cell line or may be used in transformation of a prokaryotic cell. The expression vector may exist extra-chromosomally in a transient transfectant. In a stable transfectant, the expression vector may be propagated as an episomal vector or may be integrated into the host cell chromosome. The expression vector of the present invention may further comprise at least one selectable marker gene to facilitate recognition of either prokaryotic or eukaryotic transfectants. An expression vector, as used herein, may contain both a eukaryotic and a prokaryotic origin of replication.

The term “signal peptide” as used herein refers to a hydrophobic sequence that mediates insertion of the protein through the membrane bounding the ER. Type I transmembrane proteins also comprise signal sequences. “Signal sequences,” as used herein are amino-terminal hydrophobic sequences which are usually enzymatically removed following the insertion of part or all of the protein through the ER membrane into the lumen of the ER. Thus, it is known in the art that a signal precursor form of a sequence can be present as part of a precursor form of a protein, but will generally be absent from the mature form of the protein. When a protein is said to comprise a signal sequence, it is to be understood that, although a precursor form of the protein does contain the signal sequence, a mature form of the protein will likely not contain the signal sequence. Examples of signal peptides or sequences that are functional in mammalian cells include the following: the signal sequence for interleukin-7 (IL-7) described in U.S. Pat. No. 4,965,195; the signal sequence for interleukin-2 receptor described in Cosman et al. ((1984), Nature 312:768); the interleukin-4 receptor signal peptide described in EP Patent No. 0 367 566; the type I interleukin-1 receptor signal sequence described in U.S. Pat. No. 4,968,607; the type II interleukin-1 receptor signal peptide described in EP Patent No. 0 460 846; the signal sequence of human IgG (which is METDTLLLWVLLLWVPGSTG); and the signal sequence of human growth hormone (MATGSRTSLLLAFGLLCLPWLQEGSA). Many other signal sequences are known in the art. In some embodiments, the signal peptide may be the naturally occurring signal peptide for a protein of interest or it may be a heterogonous signal peptide.

The term “binding partner” is used herein in the broadest sense and refers to two or more polypeptide sequences that are able to bind to each other under in vitro/in vivo conditions. Examples of such binding partners include, without limitation, antibody and antigen, ligand and receptor, enzyme and substrate.

As used herein, “a”, “an”, and “the” can mean singular or plural (i.e., can mean one or more) unless indicated otherwise.

### Methods of the Present Invention

The present invention provides methods of screening mammalian display libraries, including methods of screening for polynucleotides encoding a polypeptide having a desired property, methods of enriching polynucleotides encoding a polypeptide having a desired property, method of isolating a mammalian cell containing a polynucleotide encoding a polypeptide having a desired property, method of screening for mammalian cells containing a polynucleotide encoding a polypeptide having a desired property, methods of identifying a polypeptide having a desired property, and method of obtaining a polypeptide having a desired property. The methods generally entails reverse transcribing mRNA extracted from a subpopulation of mammalian cells recovered from a previous library screening into cDNA and amplifying said cDNA to generate a sublibrary of polynucleotides. This step can be repeated one or more times.

In some embodiments, the invention provides a method that comprises: a) screening an initial population of mammalian cells transfected (such as transiently transfected) with a primary library of polynucleotides for cells displaying a polypeptide having the desired property and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying a polypeptide having the desired property and recovering a second subpopulation of mammalian cells. In some embodiments, steps b) and c) are repeated more than once. In some embodiments, the method comprises: a) reverse transcribing mRNA extracted from a subpopulation of mammalian cells into cDNA and amplifying said cDNA to generate a sublibrary of polynucleotides, wherein the subpopulation of cells is obtained by screening an initial population of mammalian cells transfected (such as transiently transfected) with a primary library of polynucleotides for cells displaying a polypeptide having the desired property; b) screening a population of mammalian cells transfected with the sublibrary of polynucleotides for cells displaying a polypeptide having the desired property and recovering a second subpopulation of mammalian cells. In some embodiments, the method comprises screening a population of mammalian cells transfected with a sublibrary of polynucleotides for cells displaying a polypeptide having the desired property and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying a polypeptide having the desired property. In some embodiments, the polypeptide is a heteromeric protein. In some embodiments, the polypeptide is an antibody.

In some embodiments, the invention provides a method of isolating a polynucleotide encoding a polypeptide having a desired property, comprising: a) screening an initial population of mammalian cells transfected (such as transiently transfected) with a primary library of polynucleotides for cells displaying a polypeptide having the desired property and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying a polypeptide having the desired property and recovering a second subpopulation of mammalian cells; and d) isolating from said second subpopulation of mammalian cells a polynucleotide encoding a polypeptide having the desired property. In some embodiments, the polynucleotides in the primary library encode at least 10⁹ different polypeptides. In some embodiments, the initial population of mammalian cells are transiently transfected with the primary library under a condition where an individual mammalian cell in the population can take up more than about 100, such as more than about any of 10³, 10⁴, 10⁵, or 10⁶ copies of different polynucleotides. In some embodiments, the screening condition in step c) is more stringent than that of step a).

In some embodiments, steps b) and c) are repeated at least once prior to step d). In some embodiments, step b) and c) are repeated no more than about three times prior to step d). In some embodiments, the method further comprises transfecting (such as transiently transfecting) a primary library of polynucleotides into the initial population of mammalian cells. In some embodiments, step b) further comprises transfecting (such as transiently transfecting) said sublibrary of polynucleotides into mammalian cells. In some embodiments, the amplification of the cDNA in step b) is carried out by PCR. In some embodiments, the cDNAs are PCR amplified by using a single set of primers.

In some embodiments when steps b) and c) are repeated at least once, the assay condition in later steps can be more stringent than those in the earlier steps. A high stringency screening condition in later steps of the methods allows further enrichment in later mammalian cell subpopulations and facilitates isolation of polynucleotide.

In some embodiments, there is provides a method of isolating a polynucleotide encoding a polypeptide having a desired property, comprising: a) reverse transcribing mRNA extracted from a subpopulation of mammalian cells into cDNA and amplifying said cDNA to generate a sublibrary of polynucleotides, wherein the subpopulation of cells is obtained by screening an initial population of mammalian cells transfected (such as transiently transfected) with a primary library of polynucleotides for cells displaying a polypeptide having the desired property; b) screening a population of mammalian cells transfected with the sublibrary of polynucleotides for cells displaying a polypeptide having the desired property and recovering a second subpopulation of mammalian cells; and c) isolating from said second subpopulation of mammalian cells a polynucleotide encoding a polypeptide having the desired property.

In some embodiments, there is provides a method of isolating a polynucleotide encoding a polypeptide having a desired property, comprising: a) screening a population of mammalian cells transfected with a sublibrary of polynucleotides for cells displaying a polypeptide having the desired property and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying a polypeptide having the desired property, and b) isolating from said subpopulation of mammalian cells a polynucleotide encoding a polypeptide having the desired property.

In some embodiments, there is provided a method of screening a mammalian display library, wherein the method comprises a) screening an initial population of mammalian cells transfected (such as transiently transfected) with a primary library of polynucleotides for cells displaying a polypeptide having the desired property and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying a polypeptide having the desired property and recovering a second subpopulation of mammalian cells. In some embodiments, there is provided a method of screening a mammalian display library, wherein the method comprises: a) reverse transcribing mRNA extracted from a subpopulation of mammalian cells into cDNA and amplifying said cDNA to generate a sublibrary of polynucleotides, wherein the subpopulation of cells is obtained by screening an initial population of mammalian cells transfected (such as transiently transfected) with a primary library of polynucleotides for cells displaying a polypeptide having the desired property; b) screening a population of mammalian cells transfected with the sublibrary of polynucleotides for cells displaying a polypeptide having the desired property and recovering a second subpopulation of mammalian cells. In some embodiments, there is provided a method of screening a mammalian display library, wherein the method comprises screening a population of mammalian cells transfected with a sublibrary of polynucleotides for cells displaying a polypeptide having the desired property and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying a polypeptide having the desired property.

In some embodiments, the desired property is a specific binding to a binding partner. For example, in some embodiments, there is provided a method of isolating a polynucleotide encoding a polypeptide that recognizes a binding partner, comprising: a) screening an initial population of mammalian cells transfected (such as transiently transfected) with a primary library of polynucleotides for cells displaying a polypeptide that recognizes the binding partner and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying a polypeptide that recognizes the binding partner and recovering a second subpopulation of mammalian cells; and d) isolating from said second subpopulation of mammalian cells a polynucleotide encoding a polypeptide having the desired property. In some embodiments, step a) comprises: i) contacting the initial population of mammalian cells with a binding partner and ii) recovering a subpopulation of mammalian cells that bind to the binding partner. In some embodiments, step c) comprising: i) contacting the population of mammalian cells with the binding partner, and ii) recovering a subpopulation of mammalian cells that bind to the binding partner. In some embodiments, the condition for binding in step c) is more stringent than that of step a). In some embodiments when steps b) and c) are repeated at least once, the condition for binding in later steps can be more stringent than those in the earlier steps.

In some embodiments, the method is used to isolate polynucleotide(s) that encode a heteromeric protein. For example, in some embodiments, there is provided a method of isolating a polynucleotide that encode a heteromeric protein with a desired property, comprising: a) screening an initial population of mammalian cells for cells displaying a heteromeric protein having a desired property and recovering a subpopulation of mammalian cells, wherein the initial population of mammalian cells are transfected (such as transiently transfected) with a primary library of polynucleotides encoding a first polypeptide and a second polypeptide and wherein the first polypeptide and the second polypeptide form a heteromeric protein on the cell surface; b) reverse transcribing mRNA extracted from said subpopulation of mammalian cells into cDNA and amplifying said cDNA to generate a sublibrary of polynucleotides; c) screening a subsequent population of mammalian cells for cells displaying a heteromeric protein with a desired property and recovering a second subpopulation of mammalian cells, wherein the subsequent population of mammalian cells are transfected with the sublibrary of polynucleotides encoding a first polypeptide and a second polypeptide and wherein the first polypeptide and the second polypeptide form a heteromeric protein; and d) isolating from said second subpopulation of mammalian cells polynucleotides encoding a polypeptide having the desired property.

Polynucleotides encoding the different polypeptides in the heteromeric protein can be present in a single vector (such as a dual cassette expression vector) or on separate vectors. Thus, in some embodiments, step a) in the method described above comprises screening an initial population of mammalian cells transfected with a first primary library of polynucleotides encoding a first polypeptide and a second primary library encoding a second polypeptide. In some embodiments, the method further comprises transfecting into the initial population of mammalian cells with a first primary library encoding a first polypeptide and a second primary library encoding a second polypeptide, wherein the first polypeptide and the second polypeptide form a heteromeric protein on the cell surface. In some embodiments, the first primary library and the second primary library together encode at least 10⁹ different heteromeric proteins. In some embodiments, step b) in the method described above comprises amplifying said cDNA to generate a first sublibrary of polynucleotides encoding the first polypeptide and a second sublibrary of polynucleotides encoding the second polypeptide. In some embodiments, step b) further comprises transfecting the two sublibraries of polynucleotides into a population of mammalian cells. In some embodiments, both step a) and step c) involve mammalian cells transfected with two (or more) different libraries of polynucleotides. As discussed below in more detail, two different primer sets can be used to amplify polynucleotides encoding the first polypeptide and polynucleotides encoding the second polypeptide separately, thus allowing the cloning of these polynucleotides into different vectors.

The use of different libraries of polynucleotides at different levels of the screening cycles increases the diversity of the heteromeric proteins displayed on the mammalian cells. For example, by using a first primary library with a diversity of 10⁴ and a second primary library with a diversity of 10⁵, the total number of different heteromeric proteins that can be presented on the surface of the mammalian cells would be at least 10⁹. The diversity can be even bigger if the heteromeric protein can have more than two different polypeptide chains. Similarly, use of a first sublibrary and a second sublibrary allows shuffling to occur. For example, by combining the first sublibrary with the second sublibrary, new combinations of polypeptides in the heteromeric protein may be created. This may allow generation of heteromeric protein with a better desired property.

It can be understood by a person of ordinary skill in the art that, when steps b) and c) are repeated at least once, one round of RT-PCR can generate two or more sublibraries of polynucleotides while another round of RT-PCR can generate a single sublibrary of polynucleotides. For example, while it is desirable to use separate vectors during the early stage of the screening to increase diversity, it is sometimes preferable that a single vector encoding both the first polypeptide and the second polypeptide be used at the later round(s) of the screening so that the polynucleotide that is ultimately isolated would encode both of the two desired polypeptides. For example, in some embodiments, there is provided a method of isolating a polynucleotide that encode a heteromeric protein with a desired property, comprising: a) screening an initial population of mammalian cells for cells displaying a heteromeric protein with a desired property and obtaining a subpopulation of mammalian cells, wherein the initial population of mammalian cells are transfected (such as transiently transfected) with a primary library of polynucleotides encoding a first polypeptide and a second polypeptide and wherein the first polypeptide and the second polypeptide form a heteromeric protein on the cell surface; b) reverse transcribing mRNA extracted from said subpopulation of mammalian cells into cDNA and amplifying said cDNA to generate a first sublibrary of polynucleotides encoding a first polypeptide and a second sublibrary of polynucleotides encoding a second polypeptide; c) screening a subsequent population of mammalian cells for cells displaying a heteromeric protein with a desired property and recovering a second subpopulation of mammalian cells, wherein the subsequent population of mammalian cells are transfected with the first sublibrary of polynucleotides encoding a first polypeptide and a second sublibraries of polynucleotides encoding a second polypeptide, wherein the first polypeptide and the second polypeptide form a heteromeric protein; d) reverse transcribing mRNA extracted from said second subpopulation of mammalian cells into cDNA and amplifying said cDNA to generate a third sublibrary of polynucleotides encoding a first polypeptide and a second polypeptide, e) screening a population of mammalian cells transfected with the third sublibrary for cells displaying a heteromeric protein with a desired property and recovering a third subpopulation of mammalian cell, and f) isolating from said third subpopulation of mammalian cells polynucleotides encoding a heteromeric protein having the desired property.

As discussed further herein, the polypeptides displayed on the surface of the mammalian cells in some embodiments can be partially or completely released from the mammalian cells. This allows further analysis of the polypeptide in functional studies.

The methods described herein are applicable for the screening of any kind of polypeptides, and are particularly suitable for screening heteromeric proteins. The methods are particularly suitable, for example, for the production of protein complexes such as antibodies, T-cell receptors, class I and class II MHC molecules, integrins, CD8, CD28, and factor VIII molecules.

Also contemplated are systems and methods of screening novel heteromeric proteins with desired properties. In some embodiments, different polypeptides are fused to a dimerization domain and allowed to pair up to form heteromeric proteins. By following methods described herein, heteromeric proteins with desired properties can be identified. Thus, in some embodiments, there is provided a fusion protein comprising a heterogonous polypeptide fused to a dimerization domain. In some embodiments, the fusion protein further comprises a membrane tethering domain. The dimerization domain can be an antibody constant region. For example, in some embodiments, there is provided an antibody-like peptibody, i.e., a multimeric peptibody comprising a polypeptide fused to a light chain constant region and a polypeptide fused to a heavy chain constant region, wherein the heavy chain and light chain constant regions dimerize to produce an antibody-like molecule. For example, in some embodiments, an antibody-like peptibody contains two polypeptides fused to the light chain constant region and two polypeptides fused to the heavy chain constant region. The polypeptides in the antibody-like peptibody can be different or the same.

In the sections below, a detailed description is provided with regard to antibodies. It should be understood by a person or ordinary skill in the art, however, that the description is equally applicable to other types of polypeptides. Thus, for example, discussion about the light chain and heavy chain of the antibodies are equally applicable to the alpha and beta chains of the T cell receptor, or the different polypeptide chains of an antibody-like peptibody. It should also be understood that description below may in some embodiments also be applicable to antibody fragments (including Fab, Fab′ Fd, Fd′, Fv, dAb, isolated CDR regions; F(ab′)₂, scFv), diabodies, linear antibodies) and chimeric antibodies.

Similarly, although the description provided below focuses on isolation of polynucleotide encoding an antibody specifically recognizing an antigen, the methods are equally applicable to isolation of polynucleotides encoding a polypeptide (such as an antibody) with other desired properties, which include, for example, specific binding to a partner, higher binding affinity to a binding partner, antibody-dependent cellular cytotoxicity (ADCC), complement-dependent cytotoxicity (CDC), agonist or antagonist functions, induction or inhibition of apoptosis, angiogenesis, proliferation, activation of inhibition of signaling pathway. Multiple properties may be screened simultaneously or individually. Assay methods for these desired properties are known in the art.

### Methods of Screening Antibodies

This section describes methods of screening mammalian display libraries of antibodies in more detail. The methods include, but are not limited to, methods of screening for polynucleotides encoding an antibody having a desired property (such as specific binding to an antigen), methods of enriching polynucleotides encoding an antibody having a desired property (such as specific binding to an antigen), methods of isolating a mammalian cell containing a polynucleotide encoding an antibody having a desired property (such as specific binding to an antigen), methods of screening for mammalian cells containing a polynucleotide encoding an antibody having a desired property (such as specific binding to an antigen), methods of identifying an antibody having a desired property (such as specific binding to an antigen), and methods of obtaining an antibody having a desired property (such as specific binding to an antigen).

In some embodiments, the method comprises: a) screening an initial population of mammalian cells transfected (such as transiently transfected) with a primary library of antibodies for cells displaying an antibody having a desired property (such as specific binding to an antigen) and recovering a subpopulation of mammalian cells; b) reverse transcribing mRNA extracted from said subpopulation of mammalian cells into cDNA and amplifying said cDNA to generate a sublibrary of antibodies; c) screening a population of mammalian cells transfected with the sublibrary of polynucleotides for cells displaying an antibody having a desired property (such as specific binding to an antigen) and recovering a second subpopulation of mammalian cells. In some embodiments, steps b) and c) are repeated more than once. In some embodiments, the method comprises: a) reverse transcribing mRNA extracted from a subpopulation of mammalian cells into cDNA and amplifying said cDNA to generate a sublibrary of antibodies, wherein the subpopulation of cells is obtained by screening an initial population of mammalian cells transfected (such as transiently transfected) with a primary library of polynucleotides for cells displaying an antibody having a desired property (such as specific binding to an antigen); b) screening a population of mammalian cells transfected with the sublibrary of polynucleotides for cells displaying an antibody having a desired property (such as specific binding to an antigen) and recovering a second subpopulation of mammalian cells. In some embodiments, the method comprises screening a population of mammalian cells transfected with a sublibrary of polynucleotides for cells displaying an antibody having a desired property (such as specific binding to an antigen) and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying an antibody having a desired property (such as specific binding to an antigen). In some embodiments, the polypeptide is a full length antibody.

In some embodiments, there is provided a method of isolating a polynucleotide encoding an antibody specifically recognizing an antigen, comprising: a) screening an initial population of mammalian cells transfected (such as transiently transfected) with a primary library of polynucleotides encoding antibodies for cells displaying an antibody specifically recognizing the antigen and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying an antibody specifically recognizing the antigen and recovering a second subpopulation of mammalian cells; and d) isolating from said second subpopulation of mammalian cells a polynucleotide encoding an antibody specifically recognizing the antigen. In some embodiments, the polynucleotides in the primary library encode at least 10⁹ different antibodies.

In some embodiments, steps b) and c) are repeated at least once prior to step d). In some embodiments, step b) and c) are repeated no more than about three times prior to step d). In some embodiments, the method further comprises transfecting a primary library of polynucleotides into the initial population of mammalian cells. In some embodiments, step b) further comprises transfecting said sublibrary of polynucleotides into mammalian cells.

In some embodiments, step a) comprises: i) contacting the initial population of mammalian cells with an antigen and ii) recovering a subpopulation of mammalian cells that bind to the antigen. In some embodiments, step c) comprises: i) contacting the population of mammalian cells with the antigen, and ii) recovering a subpopulation of mammalian cells that bind to the antigen. In some embodiments, the condition for antigen binding in step c) is more stringent than that of step a). In some embodiments when steps b) and c) are repeated at least once, the condition for binding in later steps can be more stringent than those in the earlier steps.

In some embodiments, there is provides a method of isolating an antibody specifically recognizing an antigen, comprising: a) reverse transcribing mRNA extracted from a subpopulation of mammalian cells into cDNA and amplifying said cDNA to generate a sublibrary of polynucleotides, wherein the subpopulation of cells is obtained by screening an initial population of mammalian cells transfected (such as transiently transfected) with a primary library of polynucleotides for cells displaying an antibody specifically recognizing the antigen; b) screening a population of mammalian cells transfected with the sublibrary of polynucleotides for cells displaying an antibody that specifically recognize the antigen and recovering a second subpopulation of mammalian cells; and c) isolating from said second subpopulation of mammalian cells a polynucleotide encoding an antibody specifically recognizing the antigen.

In some embodiments, there is provides a method of isolating a polynucleotide encoding an antibody specifically recognizing an antigen, comprising: a) screening a population of mammalian cells transfected with a sublibrary of polynucleotides for cells displaying an antibody specifically recognizing the antigen and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying an antibody specifically recognizing the antigen, and b) isolating from said subpopulation of mammalian cells a polynucleotide encoding an antibody specifically recognizing the antigen.

In some embodiments, there is provided a method of screening a mammalian display library, wherein the method comprises a) screening an initial population of mammalian cells transfected (such as transiently transfected) with a primary library of antibodies for cells displaying an antibody specifically recognizing the antigen and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides for cells displaying an antibody specifically recognizing the antigen and recovering a second subpopulation of mammalian cells. In some embodiments, there is provided a method of screening a mammalian display library, wherein the method comprises: a) reverse transcribing mRNA extracted from a subpopulation of mammalian cells into cDNA and amplifying said cDNA to generate a sublibrary of polynucleotides, wherein the subpopulation of cells is obtained by screening an initial population of mammalian cells transfected (such as transiently transfected) with a primary library of polynucleotides for cells displaying an antibody specifically recognizing the antigen; b) screening a population of mammalian cells transfected with the sublibrary of polynucleotides for cells displaying an antibody specifically recognizing the antigen and recovering a second subpopulation of mammalian cells. In some embodiments, there is provided a method of screening a mammalian display library, wherein the method comprises screening a population of mammalian cells transfected with a sublibrary of polynucleotides for cells displaying an antibody specifically recognizing the antigen and recovering a subpopulation of mammalian cells, wherein the sublibrary is generated by reverse transcribing mRNA extracted from an intermediate population of mammalian cells into cDNA and amplifying said cDNA to generate a sublibrary of polynucleotides, wherein the intermediate population of cells is obtained by screening an initial population of mammalian cells transfected (such as transiently transfected) with a primary library of polynucleotides for cells displaying an antibody specifically recognizing the antigen.

In some embodiments, there is provided a method of isolating a polynucleotide that encodes an antibody specifically recognizing an antigen, comprising: a) screening an initial population of mammalian cells transfected (such as transiently transfected) with a primary library of polynucleotides encoding a light chain and a heavy chain for cells displaying an antibody specifically recognizing the antigen and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides encoding a light chain and a heavy chain for cells displaying an antibody specifically recognizing the antigen and recovering a second subpopulation of mammalian cells; and d) isolating from said second subpopulation of mammalian cells polynucleotides encoding an antibody specifically recognizing the antigen.

In some embodiments, there is provided a method of isolating a polynucleotide that encodes an antibody specifically recognizing an antigen, comprising: a) screening an initial population of mammalian cells transfected (such as transiently transfected) with a first primary library of polynucleotides encoding a light chain and second primary library of polynucleotides encoding a heavy chain for cells displaying an antibody specifically recognizing the antigen and recovering a subpopulation of mammalian cells; b) reverse transcribing mRNA extracted from said subpopulation of mammalian cells into cDNA and amplifying said cDNA to generate a sublibrary of polynucleotides; c) screening a population of mammalian cells transfected with the sublibrary of polynucleotides encoding a light chain and a heavy chain for cells displaying an antibody that specifically recognizing the antigen and recovering a second subpopulation of mammalian cells; and d) isolating from said second subpopulation of mammalian cells polynucleotides encoding an antibody specifically recognizing the antigen. In some embodiments, the first primary library and the second primary library allows expression and display of at least 10⁹ different antibodies on the cell surface.

In some embodiments, there is provided a method of isolating a polynucleotide that encodes an antibody specifically recognizing an antigen, comprising: a) screening an initial population of mammalian cells transfected with a first primary library of polynucleotides encoding a light chain and second primary library of polynucleotides encoding a heavy chain for cells displaying an antibody specifically recognizing the antigen and recovering a subpopulation of mammalian cells; b) reverse transcribing mRNA extracted from said subpopulation of mammalian cells into cDNA and amplifying said cDNA to generate a first sublibrary of polynucleotides encoding the light chain and a second sublibrary of polynucleotides encoding the heavy chain; c) screening a population of mammalian cells transfected with the first sublibrary of polynucleotides and the second sublibrary of the polynucleotides for cells displaying an antibody specifically recognizing the antigen and recovering a subpopulation of mammalian cells; and d) isolating from said second subpopulation of mammalian cells polynucleotides encoding an antibody specifically recognizing the antigen.

In some embodiments, there is provided a method of isolating a polynucleotide that encodes an antibody specifically recognizing an antigen, comprising: a) screening an initial population of mammalian cells transfected (such as transiently transfected) with a first primary library of polynucleotides encoding a light chain and second primary library of polynucleotides encoding a heavy chain for cells displaying an antibody specifically recognizing the antigen and recovering a subpopulation of mammalian cells; b) reverse transcribing mRNA extracted from said subpopulation of mammalian cells into cDNA and amplifying said cDNA to generate a first sublibrary of polynucleotides encoding the light chain and a second sublibrary of polynucleotides encoding the heavy chain; c) screening a population of mammalian cells transfected (such as transiently transfected) with the first sublibrary of polynucleotides and the second sublibrary of the polynucleotides for cells displaying an antibody displaying an antibody specifically recognizing the antigen and recovering a second subpopulation of mammalian cells; d) reverse transcribing mRNA extracted from said second subpopulation of mammalian cells into cDNA and amplifying said cDNA to generate a third sublibrary of polynucleotides encoding the light chain and the heavy chain; e) screening a population of mammalian cells transfected (such as stably transfected) with the third sublibrary of polynucleotides for cells displaying an antibody specifically recognizing the antigen and recovering a third subpopulation of mammalian cells; and f) isolating from said third subpopulation of mammalian cells polynucleotides encoding an antibody specifically recognizing the antigen.

### Primary Libraries of Polynucleotides Encoding Antibodies

The methods provided herein are suitable for any mammalian display libraries known in the art. Alternatively, the primary library (particularly the high complexity primary library) can be constructed with methods described herein. In some embodiments, the primary library is a naïve antibody library. In some embodiments, the primary library is an antibody library generated from individual(s) who is immunized with the antigen. In some embodiments, the primary library is a human antibody library. In some embodiments, the primary library is a library of polynucleotides encoding humanized antibodies. In some embodiments, the primary library is a chimeric antibody library. In some embodiments, the primary library is an antibody library from other mammalian species, including, but not limited to, mouse, rabbit, goat, and horse. In some embodiments, the primary library is produced from any of the immune tissues, including, for example, bone marrow, spleen, lymph nodes, lymphocytes. In some embodiments, the primary library is synthesized.

In some embodiments, the primary library comprises a first primary library of polynucleotides encoding antibody light chains and a second primary library of polynucleotides encoding antibody heavy chains. In some embodiments, the 3′ end of the heavy chain encoded by the polynucleotide is fused to a cell surface tether domain. The cell surface tether domain can be any transmembrane domain or a membrane linking sequence. Suitable cell surface tether domains include, but is not limited to, PDGFR transmembrane domain, B7-1 transmembrane domain, asialoglycoprotein receptor (ASGPR) transmembrane domain. In some embodiments, the cell surface tether domain is a GPI signal sequence which directs anchoring of the immunoglobulin to the cell-surface via a GPI linker. In some embodiments, the GPI signal sequence is from human DAF. In some embodiments, myristylation sequences can serve as the cell surface tether domain.

Most of the antibody libraries known in the art contain scFV fragment or use single vector to express both heavy chain and light chain in dual cassettes. It is feasible to build a scFV library with a diversity of 10⁹ as in phage display. It is possible to build a library with a diversity of 10⁹ to express Fab in bacteria as showed with phage display though it is more difficult and time consuming. It is doable to build a dual cassette expression library with a diversity of 10⁷ to express full-length antibody in mammalian cells though it is even much more difficult compare to phage display library. Because of the difficulties in screening mammalian cell display libraries, however, no mammalian display libraries with a diversity of 10⁹ or larger was commercially available.

The methods of the present invention makes it possible to screen mammalian display libraries with a diversity that is equal or higher than 10⁹, thus providing a reason to make mammalian display libraries with such high diversity. Accordingly, the present invention also provides a method of constructing a library of polynucleotides encoding antibodies, wherein the polynucleotides collectively encode at least about 10⁹, including for example at least about 10¹⁰, 10¹¹, 10¹², 10¹³, 10¹⁴, or 10¹⁵ different recombinant antibodies. Each polynucleotide in the library could encode both a light chain and a heavy chain. Alternatively, a first library of polynucleotide encoding a light chain can be combined with a second library of polynucleotide encoding a heavy chain. Once transfected into a mammalian cell, the heavy chain and the light chain can be paired inside the cell, thus creating a high diversity of different antibodies. Accordingly, in some embodiments, there is provided a method of constructing a library of polynucleotides encoding antibodies comprising combining a library of light chain with a library of heavy chain such that the polynucleotides collectively encode at least about 10⁹ different recombinant antibodies.

In some embodiments, the primary library is derived from a single individual. Accordingly, in some embodiments, there is provided a method of constructing a library of polynucleotides encoding antibodies from a single individual, wherein the polynucleotides collectively encode at least about 10⁹, including for example at least about 10¹⁰, 10¹¹, 10¹², 10¹³, 10¹⁴, or 10¹⁵ different recombinant antibodies. Accordingly, in some embodiments, there is provided a method of constructing a library of polynucleotides encoding antibodies from a single individual, comprising combining a library of light chain of the individual with a library of heavy chain of the individual such that the polynucleotides collectively encode at least about 10⁹ different recombinant antibodies.

In some embodiments, the polynucleotides are present in plasmid vectors. In some embodiments, there is provided a population of mammalian cells displaying at least about 10⁹, including for example at least about 10¹⁰, 10¹¹, 10¹², 10¹³, 10¹⁴, or 10¹⁵ different recombinant antibodies on the surface. In some embodiments, there is provided a population of mammalian cells transiently transfected with a library of polynucleotides, wherein the polynucleotides collectively encode at least about 10⁹, including for example at least about 10¹⁰, 10¹¹, 10¹², 10¹³, 10¹⁴, or 10¹⁵ different recombinant antibodies. In some embodiments, there is provided a population of mammalian cells transiently transfected with a first library of polynucleotides encoding an antibody light chain and a second library of polynucleotides encoding an antibody heavy chain, wherein the polynucleotides collectively encode at least about 10⁹, including for example at least about 10¹⁰, 10¹¹, 10¹², 10¹³, 10¹⁴, or 10¹⁵ different recombinant antibodies. In some embodiments, there are provided methods of screening a population of mammalian cells described above for polynucleotides encoding antibodies of a desired property (such as specific binding to an antigen).

Also provided in the present invention are methods of constructing a library of polynucleotides encoding antibody-like peptibodies, wherein the polynucleotides collectively encode at least about 10⁹, including for example at least about 10¹⁰, 10¹¹, 10¹², 10¹³, 10¹⁴ or 10¹⁵ different recombinant peptibodies. In some embodiments, there is provided a population of mammalian cells displaying at least about 10⁹, including for example at least about 10¹⁰, 10¹¹, 10¹², 10¹³, 10¹⁴ or 10¹⁵ different recombinant antibody-like peptibodies on the surface. In some embodiments, there is provided a population of mammalian cells transiently transfected with a library of polynucleotides, wherein the polynucleotides collectively encode at least about 10⁹, including for example at least about 10¹⁰, 10¹¹, 10¹², 10¹³, 10¹⁴ or 10¹⁵ different recombinant antibody-like peptibodies. As discussed above, although the discussion herein focuses primarily on antibodies, the description, whenever applicable, would apply to antibody-like peptibodies as well. The present application also encompasses methods of screening a library of antibody-like peptibodies as described herein.

There are a variety of techniques that may be used to efficiently generate libraries of immunoglobulins, including those described or referenced in Molecular Cloning; A Laboratory Manual, 3rd Ed. (Maniatis, Cold Spring Harbor Laboratory Press, New York, 2001), Current Protocols in Molecular Biology (John Wiley &amp; Sons), U.S. Pat. No. 6,403,312, U.S. Ser. No. 09/782,004, U.S. Ser. No. 09/927,790, U.S. Ser. No. 10/218,102, PCT WO 01/40091, and PCT WO 02/25588, each of which is incorporated by reference in its entirety.

Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. A variety of commercially available kits and methods for gene assembly, mutagenesis, vector subcloning, and the like, are available for generating nucleic acids that encode immunoglobulin amino acid sequences.

Vectors for expressing antibodies (particularly full length antibodies) on the surface of mammalian cells can be prepared according to methods known in the art. In some embodiments, the polynucleotide encoding an immunoglobulin heavy chain constant region is fused as its 3′ end to a polynucleotide encoding the cell surface tether domain, allowing display of the expressed immunoglobulins on the surface of the mammalian cells. In some embodiments, there is an enzymatic cleavage site between the heavy chain constant region and the membrane tether domain, the cleavage of which allows the immunoglobulin be removed enzymatically, thus enabling conversion of the expressed immunoglobulin from a membrane-bound form to a soluble form. The cleaved antibody can be used directly for further functional analysis, thus obviating the step of converting a membrane anchored form into a soluble form.

In some embodiments, there is a furin cleavage site between the heavy chain constant region and the cell surface tether domain. Furin is a protease which resides in the trans-Golgi network of eukaryotic cells. Its function is to cleave proteins at a step just prior to their delivery to their final cellular destination. Furin recognizes a consensus amino acid sequence, RXRR (SEQ ID NO:1), RXRK (SEQ ID NO:2), or KXKR (SEQ ID NO: 3)(where X is any amino acid) and cuts proteins which contain these sequences when they reach the transgolgi network. See U.S. Pat. No. 7,223,390; Poole et al., J. Biol. Chem. 2003, Vol. 278(38):36183-36190; Chen et al., 2001, Proc. Natl. Acad. Sci., vol. 98(13):7218-7223. The expressed protein containing the furin cleavage site typically goes through its normal process of folding and assembly to attain its native configuration. After export from the endoplasmic reticulum, the protein travels along the secretory pathway to reach the cell surface. When the protein reaches the trans-Golgi network, it is cleaved by the furin protease. Because Furin cleavage is partially complete, a portion of the antibodies will be released into the medium while the other portion of the antibodies will remain attached to the cell membrane. This configuration allows functional assays of the cleaved antibody while maintaining the link between the antibody and the cell expressing the antibody.

Suitable promoters for expression of the recombinant polypeptides in the mammalian cells include, but are not limited to, SV40 promoter, mouse mammary tumor virus promoter, human HIV long terminal repeat promoter, moloney virus promoter, avian leukemia virus promoter, Epstein-Barr virus immediate early promoter, a Rous sarcoma virus promoter, human actin promoter, human hemoglobin promoter, CMV promoter, human EF-1 alpha promoter, or human muscle creatine promoter.

In some embodiments, the vector comprises a first promoter operatively linked to the first polynucleotide encoding an immunoglobulin heavy chain and a second promoter operatively linked to the second polynucleotide encoding an immunoglobulin light chain. In some embodiments, the immunoglobulin light chain and heavy chain are present in two separate vectors, each having a different promoters operably linked to the immunoglobulin light chain and heavy chains.

### Mammalian Cells

The mammalian cells for the library screening can be derived from any eukaryotic species, including but not limited to cells from rat, mouse, bovine, porcine, sheep, goat, and human. The cells can be maintained according to standard methods well known to those of skill in the art.

Examples of suitable mammalian cells include HeLa cells (HeLa S3 cells, ATCC CCL2.2), Jurkat cells, Raji cells, Daudi cells, human embryonic kidney cells (293-HEK; ATCC 293c18, ATCC CRL 1573), African green monkey kidney cells (CV-I; Vero; ATCC CRL 1587), SV40-transformed monkey kidney cells (COS-I; ATCC CRL 1650), canine kidney cells (MDCK; ATCC CCL 34), baby hamster kidney cells (BHK-21, BHK-570; ATCC CRL 8544, ATCC CRL 10314), Chinese hamster ovary cells (CHO-Kl; ATCC CCL61; CHO DG44 (Chasin et al., 1986, Som Cell Molec Genet, 12, 555)), and other rodent cell lines such as NS0, SP2/0, GH1 (ATCC CCL82), H-4-II-E (ATCC CRL 1548), NIH-3T3 (ATCC CRL 1658). In some embodiments, the mammalian cell is selected from the group consisting of 293-HEK, Hela, Jurkat, Raji, Daudi, COS, or CV-1 cells. In some embodiments, the mammalian cells are derivatives of the cells described above.

Mammalian cells can be transformed with polynucleotides using suitable means and cultured in conventional nutrient media modified as is appropriate for inducing promoters. Representative examples of such methods include transfection using calcium phosphate precipitation, lipid mediated transfection, direct microinjection of polynucleotides into intact target cells, and electroporation. In some embodiments, the mammalian cells are transiently transfected.

Suitable culture conditions for cells, such as temperature and pH, are well known. The mammalian cells used in different steps of the methods described herein can be the same or different. It should be noted that, while in traditional methods of screening it is desirable (and sometimes critical) to control the copies of vectors taken up by a cells, during the initial steps of the methods in the present invention it is preferable that each mammalian cell can take up as many vector copies as possible. For example, the transfection condition may be optimized in such a way that each mammalian cell takes up at least about 20, such as at least about any of 50, 100, 200, 300, 400, 500, 1000, or more plasmids up to 10⁶, 10⁷ per cell. The transfection conditions can be optimized by adjusting parameters such as cell density, concentration of vector DNA, ratio of the different primary libraries (when applicable), transfection reagents, and transfection procedure.

Accordingly, the present invention in one aspect provides a population of mammalian cells, at least some (for example 50%, 60%, 70%, 80%, 90%, 99%) of which comprise least about 20, such as at least about any of 50, 100, 200, 300, 400, 500, 1000, or more different polynucleotides up to 10⁶ or 10⁷ per cell. In some embodiments, the mammalian cells are transiently transfected.

### Initial Screening

Following introduction of the polynucleotide libraries into cells, the antibodies are allowed to be displayed on the cell surface of the mammalian cells. By “allowing display” is meant allowing the vectors which have been introduced into the cells to undergo transcription and translation of the polypeptides and transporting the antibodies to the membrane surface. The conditions, and time required to allow expression will vary depending on the choice of the host cell and the choice of vectors, as is well known by one of ordinary skill in the art.

In some embodiments, cells expressing antibodies on their surface are subsequently contacted with an antigen by a method which will allow the antigen to bind to the desired antibody, thereby allowing the cells expressing the antibody to be distinguished from those cells which do not bind the antigen. The initial screening step allows a first subpopulation of mammalian cells be recovered.

“Recovery” is meant separation of a desired component from those components which are not desired. It should be understood by a person in the ordinary skill in the art that, although the subpopulation of mammalian cells are enriched with cells displaying an antibody specifically recognizing an antigen, most cells in the subpopulation are likely to be non-specific binders. In some embodiments, the initial screening is carried out in a low stringency condition to maximize the possibility of capturing cells displaying an antibody that specifically recognize the antigen.

The initial screening can be carried out using any one of the following methods: fluorescence-activated cell sorting (FACS), bead-based sorting such as magnetic bead-based sorting (MACS), a combination thereof, or other solid-phase panning techniques. Other techniques that can be used are also known in the art. For example, if the cells are in suspension, and the antigen is attached to a solid substrate, cells which specifically bind to the antigen will be trapped on the solid substrate, allowing those cells which do not bind the antigen to be washed away, and the bound cells can subsequently be recovered. Alternatively, if the cells are attached to a solid substrate, and by specific binding to the antigen are caused to be released from the substrate, they can be recovered from the cell supernatant.

The antigen can be attached to the solid substrate (such as magnetic beads) either directly or indirectly. For example, in some embodiments, the solid substrate can be coated by a streptavidin, which allows antigens linked to a biotin be attached to the solid substrate via the interaction of streptavidin and the biotin. Other binding pairs can be also be used.

In some embodiments, the antigen can be expressed and presented at the surface of a cell. The interaction between the antigen presenting cell and the cells that bind to the antigen allows separation of the cells binding to the antigen (for example, cells expressing the desired antibody). For example, the cell complexes can be isolated by ways of FACS. In some embodiments, the interaction between the antigen presenting cells and the cells that bind to the antigen could induce a detectable signal (either within the antigen presenting cells or the cells that bind to the antigen) that allows separation of the cells binding to the antigen.

In some embodiments, the cells are incubated with an antigen that has been labeled directly or indirectly with a fluorescent label (such as fluorescein-5-isothiocyanate (FITC)). Cells expressing an antibody binding to the fluorescence tagged antigen can be selected by fluorescence activated cell sorting (FACS), thereby permitting the cells which specifically bind the antigen to be distinguished from those cells which do not bind the antigen.

In some embodiments, one or more of the above-techniques can be combined. For example, solid phase panning can be combined with the use of FACS or vice versa.

In some embodiments when a desired affinity is used as a basis for screening, ELISA assays can be used to determine the binding affinity of an isolated immunoglobulin toward a target antigen.

As discussed above, in some embodiments, the antibody can be partially or completely cleaved from the fused tether domain and can be used to further assay and/or confirm the desired properties.

### Generation and Screening of Sublibrary

The methods described herein encompass methods of generating and screening sublibraries as described herein. After recovery of the initial subpopulation of mammalian cells, total mRNA is extracted from the cells. The mRNA is transcribed into cDNA, and the antibody genes or parts the antibody genes (such as the VH and VL regions of the antibody genes) are amplified (for example by PCR).

Unlike traditional methods of generating primary antibody libraries where a mixture of primers are needed for amplification of the antibody variable regions, primers used for polynucleotides in the sublibrary can be based on vector sequence of the primary library. For example, primers can be designed based on sequences in the vectors to amplify both the light chain and the heavy chain sequences.

In some embodiments, the VH and VL regions of the antibody sequence is cloned into the backbone vectors containing CH and CL regions respectively to construct two sub-libraries, heavy and light chain sub-libraries. The two sublibraries are subsequently transfected into mammalian cells. This step allows shuffling of the heavy and light chain in cells, which may increase the chance to isolate an antibody with higher expression as well as higher affinity. In such embodiments, at least one primer for amplifying the light chain is different from the one used for amplifying the heavy chain so that the light chain and heavy chain can be separately cloned into different vectors, and divided into different sublibraries.

The amplified antibody sequences (such as the VH and VL sequences) can be cloned back into the same vectors used in the primary library or into a different vector. For example, in some embodiments, the primary library comprises a primary light chain library and a different second primary heavy chain library, with the light chain and heavy chains on different vectors (such as plasmid vectors). After the initial screening, the amplified light chain and heavy chain sequences are then cloned into a dual cassette single vector. In some embodiments, the vector is similar to the vector used in Higuchi et al., J. Immun. Methods (1997) 202:193-204. In some embodiments, separate libraries were used for several rounds of screening before cloning the light and heavy chains into a single dual expression vector. Cloning dual expression vector at the final stage of the screening allows simultaneous identification of the pair of antibody light chain and heavy chains for an antibody with the desired property (such as specific binding to an antigen).

The mammalian cells used in the subsequent screening steps may be of the same type used in the first round of screening, or may be different cells, as long as they are capable of expressing the polypeptide on the cell surface. The screening can be carried out using any of the above methods described for the initial screening.

The RT-PCR and screening steps described herein can be repeated at least one, two, three, four, five, six, or seven times. In some embodiments, the steps are repeated no more than about three times. The stringency for the different cycles of screening can be the same or different to facilitate the isolation of the polynucleotide. For example, a higher stringency condition is used towards the end of the screening cycle to decrease the diversity of the polynucleotides obtained from the enriched subpolulations. The diversity of the sublibraries can be evaluated during the various steps of the method. A diversity of less than about 1000 to 10000 signals that an isolation step can be carried out as described below.

### Isolation of Polynucleotide Encoding an Antibody Specifically Recognizing the Antigen

To isolate the antibody sequence, the total mRNA is isolated from the final sub-cell pool. The relevant regions are RT-PCR amplified, restriction enzyme digested and cloned into a stable expression vector. The DNA is then stably transfected into mammalian cells under conditions that most mammalian cells have only one expression vector in each cell. For example, a titration step can be used to dilute the concentration of plasmid used for cellular transfection to reduce the likelihood of expression in the same cell of multiple vectors encoding different immunoglobulins. Alternatively, a flip-in system, e.g., FLP-In™ System (Invitrogen, Inc.) can be used.

Cells expressing the desired antibody can be isolated, for example, by methods described above. The polynucleotide encoding the desired antibody can be isolated and sequenced.

### Single-Cassette Expression Vectors and Dual Cassette Expression Vectors

The present invention also provides specific expression vectors for use in methods described herein. These expression vectors provide high cloning efficiency, and are particularly useful for constructions of high complexity protein libraries such as high complexity antibody libraries described herein, as well as for use in methods of screening libraries, such as methods described herein.

The vectors provided herein can be used for rapid construction of any antibody libraries, heavy chain, light chain, chimerical and even fusion proteins, and for high expression of these antibody proteins on the mammalian cell surface for efficient screening and selecting (for example, when coupled with FACS). Two or more unique endonuclease recognizing sequences can be incorporated into the vector for the pop-in and pop-out of genes of interest. Using such vector a library with a size of at least 10⁶ complexity can be readily constructed. CMV promoter, a commonly used promoter for high expression of proteins in variety of mammalian cells, can be used to drive the expression of inserted genes (such as antibody genes). To anchor the antibody expressed on the mammalian cell surface, a trans-membrane domain from PDGFR can be fused in frame to the C-terminal of heavy chain consistent region.

Further provided herein are dual cassette expression vectors. Such vectors can contain dual mammalian expression cassettes for the one-step insertion of both heavy chain and light chain genes to display full-length bivalent antibodies on mammalian cell surfaces. This can be achieved by fusing a transmembrane domain from PDGFR to the C-terminus of the heavy chain constant region. We can also incorporate a furin cleavage site between the constant region and PDGFR transmembrane domain to obtain secreted antibodies. As a result, antibodies can be expressed simultaneously on the cell surface in a membrane-anchored version for screening and selecting through FACS analysis as well as in condition medium in a secreted version for function analysis. Using this procedure, we could rapidly construct an antibody library with a size of 10⁶ in only a few days. This novel one-step dual-expression mammalian display system is particularly useful for the rapid screening of biologically active human monoclonal antibodies with high affinity.

Thus, in some embodiments, there is provided an expression vector comprising an open reading frame flanked by a pair of cleavage sites recognizable by a restriction enzyme (for example a restriction enzyme that recognizes a non-palendromic sequence), wherein the ends of each fragment resulting from the cleavage with said restriction enzyme do not self-ligate. In some embodiments, the open reading frame encodes an antibody heavy chain. In some embodiments, the antibody heavy chain is fused to a transmembrane domain. In some embodiments, there is an enzyme cleavage site (such as furin cleavage site) between the antibody heavy chain and the transmembrane domain. In some embodiments, the open reading frame encodes an antibody light chain.

In some embodiments, the vector further comprises a second pair of cleavage sites recognizable by a different restriction enzyme (such as restriction enzymes that recognizes a non-palendromic sequence), wherein the ends of each fragment resulting from the cleavage with said second restriction enzyme do not self-ligate. In some embodiments, the second pair of cleavage sites flank the open reading frame. In some embodiments, one of the second pair of cleavage sites flanks the open reading frame wherein the other one of the second pair of cleavage sites is located within the open reading frame. For example, in some embodiments, the open reading frame comprises an antibody heavy chain, and one of the second pair of cleavage sites is located between the variable region and the constant region. Such vector may be suitable, for example, for cloning an antibody variable region. Similarly, in some embodiments, the open reading frame comprises an antibody light chain, and one of the second pair of cleavage sites is located between the variable region and the constant region. Such vector may be suitable, for example, for cloning an antibody variable region.

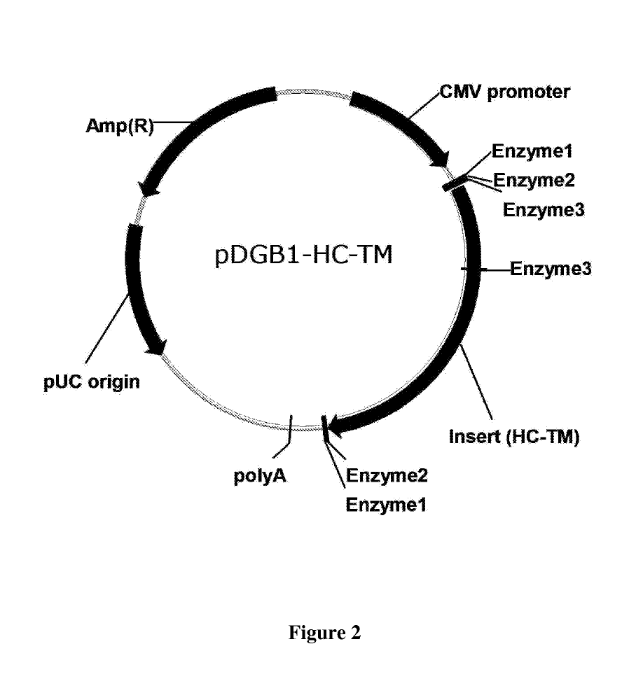

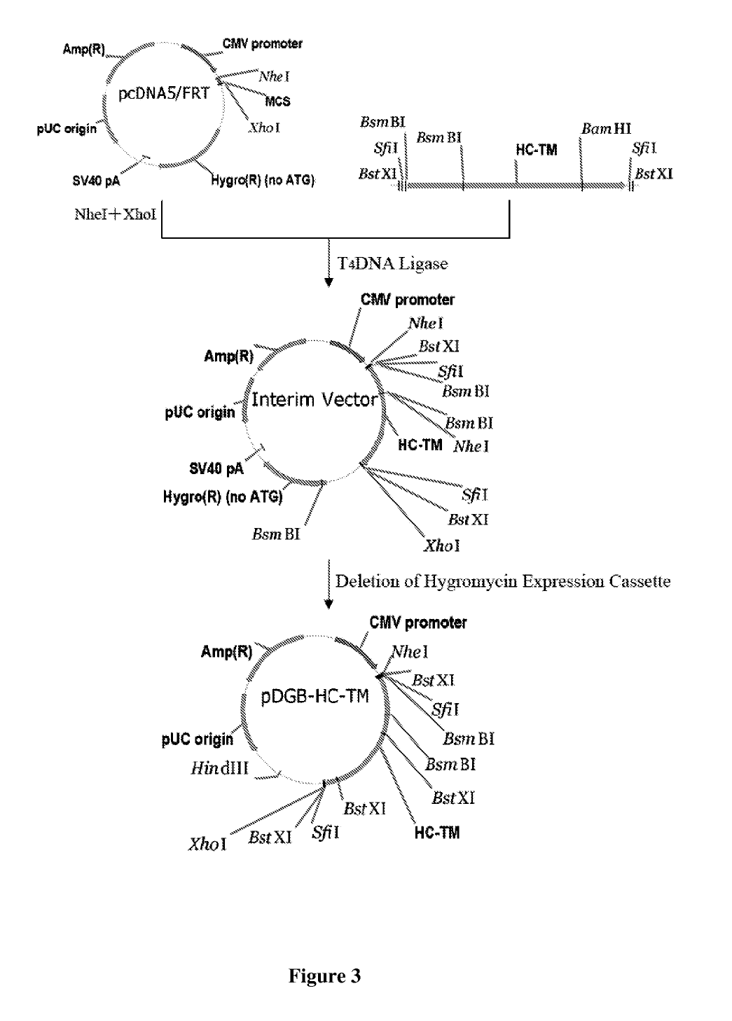

In some embodiments, the restriction enzyme is selected from the group consisting of SfiI, BstXI, and BsmB1. It is to be understood that other enzymes with similar properties can be used as well. In some embodiments, the vector further comprises a promoter that is operably linked to said open reading frame. In some embodiments, the vector has a restriction map as depicted in FIG. 2. In some embodiments, the vector has a restriction map as depicted in FIG. 3.

In some embodiments, the expression vector comprises a second open reading frame. Thus, for example, in some embodiments, there is provided a dual-expression cassette vector, comprising: 1) a first open reading frame flanked by first pair of cleavage sites recognizable by a restriction enzyme (such as a restriction enzyme that recognizes a non-palendromic sequence), wherein the ends of each fragment resulting from the cleavage with said restriction enzyme do not self-ligate; and 2) a second open reading frame flanked by a second pair of cleavage sites recognizable by a restriction enzyme (such as a restriction enzyme that recognizes a non-palendromic sequence), wherein the ends of each fragment resulting from the cleavage with said second restriction enzyme do not self-ligate. In some embodiments, one of the open reading frames encodes an antibody heavy chain. In some embodiments, the antibody heavy chain is fused to a transmembrane domain. In some embodiments, there is an enzyme cleavage site (such as furin cleavage site) between the antibody heavy chain and the transmembrane domain. In some embodiments, one of the open reading frames encodes an antibody light chain. Thus, for example, in some embodiments, there is provided a dual-expression cassette vector, comprising: 1) a first open reading frame encoding an antibody heavy chain, flanked by first pair of cleavage sites recognizable by a restriction enzyme (such as a restriction enzyme that recognizes a non-palendromic sequence), wherein the ends of each fragment resulting from the cleavage with said restriction enzyme do not self-ligate; and 2) a second open reading frame expressing an antibody light chain, flanked by a second pair of cleavage sites recognizable by a restriction enzyme (such as a restriction enzyme that recognizes a non-palendromic sequence), wherein the ends of each fragment resulting from the cleavage with said second restriction enzyme do not self-ligate. Also provided are fragments resulting from said cleavage, which are useful for cloning desired sequences (such as antibody sequences) by way of four-way ligation. Also provided are methods of using the dual-cassette expression vector for expressing full-length antibodies, and use of said vector for cloning desired sequences.

In some embodiments, the dual-cassette expression vector further comprises a third pair of restriction enzyme cleavage sites recognizable by a third restriction enzyme (such as a third restriction enzyme that recognizes a non-palendromic sequence), wherein the ends of each fragment resulting from the cleavage with said third restriction enzyme do not self-ligate. In some embodiments, one of the third pair of cleavage sites flanks the one of the open reading frames on the vector, and wherein the other one of the third pair of cleavage sites is located within the same open reading frame. For example, in some embodiments, one of the open reading frames comprises an antibody heavy chain, and one of the third pair of cleavage sites is located between the variable region and the constant region. Similarly, in some embodiments, one of the open reading frames comprises an antibody light chain, and one of the third pair of cleavage sites is located between the variable region and the constant region.

In some embodiments, the dual-cassette expression vector further comprises a fourth pair of restriction enzyme cleavage sites recognizable by a third restriction enzyme (such as a third restriction enzyme that recognizes a non-palendromic sequence), wherein the ends of each fragment resulting from the cleavage with said third restriction enzyme do not self-ligate. In some embodiments, one of the fourth pair of cleavage sites flanks the one of the open reading frames on the vector, and wherein the other one of the fourth pair of cleavage sites is located within the same open reading frame. It is contemplated that the third pair of cleavage site and the fourth pair of cleavage site can each flank (and locate within) a different open reading frame in the dual cassette expression vector. For example, in some embodiments, one of the open reading frame comprises an antibody heavy chain, and one of the third pair of cleavage sites is located between the variable region and the constant region; whereas one of the open reading frame comprises an antibody light chain, and one of the fourth pair of cleavage sites is located between the variable region and the constant region.

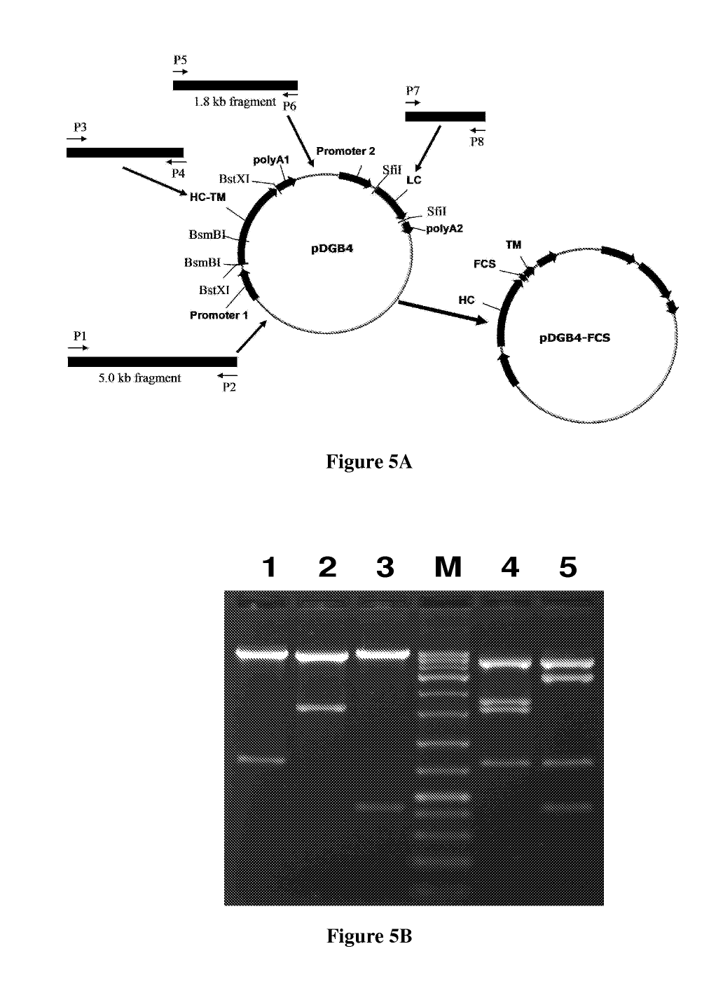

In some embodiments, the restriction enzyme is selected from the group consisting of SfiI, BstXI, and BsmB1. In some embodiments, the vector further comprises a first promoter operably linked to said first open reading frame and a second promoter operably linked to said second open reading frame. In some embodiments, the vector has a restriction map as depicted in FIG. 5A.

### Kits, Libraries, Sublibraries, Polynucleotides, and Polypeptides

Also provided are libraries and kits for carrying out the methods described herein. Further provided are sublibraries generated during the screening process and polynucleotides/polypeptides obtained from methods described herein.

In some embodiments, there is provided a library of polynucleotides encoding an antibody light chain and a library of polynucleotides encoding an antibody heavy chain, wherein the two libraries allow expression of at least 10⁹, including for example at least any of 10¹⁰, 10¹¹, 10¹², 10¹³, 10¹⁴, 10¹⁵, 10¹⁶, 10¹⁷, or more different antibodies. In some embodiments, the antibody light chain comprises: (i) a light chain constant region, (ii) an immunoglobulin light chain variable region, and (iii) a signal peptide. In some embodiments, the antibody heavy chain comprises: (i) at least one antibody heavy chain constant region, (ii) an immunoglobulin heavy chain variable region, and (iii) a signal peptide. In some embodiments, the antibody heavy chain further comprises a cell surface tethering domain at the C-terminal end of the heavy chain constant region. In some embodiments, the antibody heavy chain further comprises a furin cleavage site between the heavy chain constant region and the cell surface tethering domain.

In some embodiments, the two libraries are provided in a kit, either in separate containers or in the same container. In some embodiments, the kit further comprises primers, reagents, cells, and instructions for carrying out methods described herein. For example, in some embodiments, there is provided a kit comprising 1) a first plasmid library of polynucleotides encoding a plurality of immunoglobulin heavy chains, 2) a second plasmid library of polynucleotide encoding a plurality of immunoglobulin light chains. In some embodiments, the kit further comprises a population of cells capable of expressing immunoglobulin molecules. In some embodiments, the kit further comprises enzymes and primers for RT-PCR. In some embodiments, the first and the second plasmid libraries are contained in separate containers. In some embodiments, the kit further provides an instruction for carrying out the one or more methods describe herein.

In some embodiments, there is provided a kit comprising 1) a set of primers for the generation of primary libraries (such as primary libraries of antibodies), 2) a set of vectors for transient and/or stable transfection of mammalian cells; and 3) a set of primers for the generation of sublibraries (such as sublibraries of antibodies). In some embodiments, the kit further provides an instruction for carrying out the methods describe herein.

Also provided are polynucleotides obtained for methods described herein and polypeptides encoded by such polynucleotides. The polynucleotides and polypeptides can be useful, for example, as research reagents, diagnostic reagents and therapeutics for treatment of diseases.

Also provided are sublibraries generated by the RT-PCR methods described herein. For example, in some embodiments, there is provided a sublibrary of polynucleotides encoding a plurality of polypeptides, wherein the sublibrary is generated by reverse transcribing mRNA extracted from a population of mammalian cells into cDNA and amplifying said cDNA, wherein the population of cells is obtained by screening an initial population of mammalian cells transfected (such as transiently transfected) with a primary library of polynucleotides for cells displaying a polypeptide having the desired property.

Further provided are kits comprising expression vectors, such as the single cassette and dual cassette expression vectors described herein. The kit can either comprise the entire vector, or linear fragments of the vector which can directly be used for ligation (such as four-way ligation).

The following Examples are provided to illustrate, but not limit, the invention.

### Example 1

This example shows construction of a vector which can express antibody in mammalian cell in both surface anchored version and secreted version simultaneously.

In order to express antibodies from mammalian cells for screen, it is important to design and construct a specific expression vector which can:

1. express antibody in mammalian cells at a level high enough for detection and analysis;

2. express antibody molecules both on mammalian cell surface and in a secret version;

3. be amplified in and purified from bacteria easily;

4. be delivered into mammalian cells with high efficiency

For this purpose, the vector pcDNA 3.1 from Invitrogen is used as the starting material. The vector size is 5.4 kb, containing most necessary components for expression of proteins in mammalian cells. To increase transfection efficiency (copy number per cell point of view), the 1.8 kb Neo gene expression cassette is deleted to reduce the vector size. To facilitate the insertion of the genes of interest, the MCS (multiple-cloning-site) is modified to include BstX I and Sal I. The suitable version of vector has a site of about 3.2 kb.

This 3.2 kb vector is used as backbone vector for construction of antibody expression library. When the gene of interest is inserted into this vector in MCS, the gene is expressed when the vector is delivered into mammalian cells.

To express displayed heavy chain, the vector is further modified by insertion of PDGFR coding sequence at the 3′-end of heavy chain constant region.

To express antibodies in both displayed and secreted versions simultaneously, a Furin cleavage site (FCS) is inserted between heavy chain and PDGFR.

Following figure shows the relationship of components in heavy chain in expression vector:

Promoter----V------- C-------- FCS--PDGFR

### Example 2

This example describes the construction of a vector for stable express of antibody in mammalian cell in both surface anchored version and secreted version simultaneously.

For expression of both heavy and light chain from single vector, dual expression vector is constructed. One more expression cassette is inserted into vector pcDNA3.1. The Ch and Cl gene sequences are inserted into this vector at separate cassettes. The relationship of these two cassettes are showed as follows, where the promoter 1 and promoter2 are the same or different.

Promoter1---Ch or Cl---PolyA----Promoter2---Cl or Ch---PolyA----Selection
marker

To express cell surface displayed antibody, the vector is further modified by insertion of PDGFR coding sequence at the 3′-end of heavy chain constant region.

To express antibodies in both displayed and secreted versions simultaneously, a Furin cleavage site (FCS) is inserted between heavy chain and PDGFR.

### Example 3

This example describes construction of full-length human antibody mammalian display library.

This example shows the building of a full-length human antibody mammalian display library with a diversity of 10⁹ or larger. The vector constructed in example 1 is used as the backbone for construction of the library.

The genes of variable domain of human antibody heavy chain and light chain (Vh and Vl) are amplified by PCR from ready-to-use cDNA of human immune tissue (bone marrow, spleen and peripheral blood lymphocytes) (BioChain, South San Francisco, Calif. USA), using primers as described in book Phage Display (Carles et al, Cold Spring Harbor Laboratory Pr.). The PCR conditions are optimized to ensure the efficient and accurate amplification of Vh and Vl. The PCR products are digested with suitable restriction enzymes matched with cloning vector. After digestion the products are purified using PCR purification kit (Qiagen).

For heavy chain expression, the heavy chain constant region (Ch) of IgG1, FCS and PDGFR sequence are cloned into the vector from example 1 in MCS. Then the Vh mixture is inserted into the vector in frame before Ch region using T4 ligase. After transformation of ligation mixture, the transformation efficiency and library size are calculated by measuring the transformant number to make sure the library size is 10e5 or larger. The diversity of library is analyzed and calculated more accurately by sequence analysis of more than 100 individual clones. The FCS region may not be included in the vector if the antibody is only need to be expressed on cell surface.

For light chain expression, the kappa (or lambda) chain constant region (Cl) is cloned into the vector from example 1 in MCS. Then the Vl mixture is inserted into the vector in frame before Cl region using T4 ligase. After transformation of ligation mixture, the transformation efficiency and library size are calculated by measuring the transformant number to make sure the library size is 10e4 or larger. The diversity of library will be analyzed and calculated more accurately by sequence analysis of more than 100 individual clones.

Both heavy chain and light chain library vectors can be easily amplified in bacteria and stored in −20 C with safe.

This library is a primary, naïve full-length human antibody library with a potential diversity of >=10e9. The antibodies expressed from this library can be anchored on mammalian cell surface for affinity and function screening and selecting directly. At the same time, part of the antibodies are expressed as soluble molecules into condition medium, which can be used directly for further function analysis and characteristic analysis of antibodies without the conversion of antibodies from membrane anchored format to soluble format, speeding up the screening and selecting.

### Example 4

This example describes the expression and screening of mammalian cell surface displayed antibody library and selecting antibody candidates

The libraries constructed from Example 3 are vector DNA and can be amplified. To amplify the vector library, the vector DNAs are transformed into bacteria competent cells. Antibiotic-resistant colonies are collected and vector DNAs are purified from bacteria using max-prep kit (Qiagen).

293 cells are cultured in DMEM medium (Invitrogen) supplemented with 10% FBS without antibiotics. The transfection conditions are optimized in terms of cell density, vector DNA dose, the ratio of heavy and light chain libraries, transfection reagent and transfection procedure using transfection optimization kit (Biocompare, S. San Francisco, Calif.).

To prepare cells for transfection, the 293 cells are splited as need and seeded into T225 flasks coated with 1% poly-lysine.

Using optimized conditions, the DNA library mixture is transfected into 293 cells in T225 flasks. 48-72 hours post-transfection, the cells are dissociated using dissociate buffer (Invitrogen) and suspended in staining buffer (PBS with 2% FBS) in a density of 1e6-1e7/ml. The cells are double-stained with PE-labeled mouse anti-human IgG1 heavy chain and FITC-labeled anti-human kappa chain constant region antibodies (1 ug/ml). Then the expression of antibodies on the cell surface is analyzed by FACS. Usually more than 90% of the cells are stained positively compared to negative control. This is the primary cell library.

To screen the library for specific antibodies, the selected antigen (for specificity, see other examples) is labeled with biotin, then used to staining the antibody positive cell population as conformed as described above.

The streptavidin-conjugated magnetic beads (Dynal/Invitrogen) are mixed with antigen-stained cell population. The specific antibody positive cells are captured by magnetic beads and collected by magnetic field as described in product manual.

Because the percentage of specific antibody genes is very low, the number of cells expressing specific antibody is rare in the cell pool. In this step, low stratingen binding conditions are applied to ensure that all of the binder to the antigen can be captured. Here the purpose is to enrich the binder and the purity is ignored. Even the purity is only one out of ten thousands ( 1/10e4), it has already been enriched at least 10e5 folds comparing to the library diversity of =>10e9 in one cycle of enrichment.

Following steps are important for the success in isolating antigen specific antibodies from highly diversified library because even though the above enrichment may already be up to 10e5 folds, most of the cells in the pool are expressing non-specific antibodies.

1. To further enrich the specific binders, extract total mRNA from the sub-cell pool of above enrichment step. The Vh and Vl regions of the antibody genes in the total mRNA are RT-PCR amplified and cloned into the backbone vectors containing Ch and Cl regions respectively to construct two sub-libraries, heavy and light chain sub-libraries.

2. Transfect the mixture of these sub-libraries into fresh 293 cells and analyze the antibody expression on cell surface.

3. Enrich the specific antigen binders as done for primary cell library.

Repeat above steps 1 to 3 as many cycles as needed. The stringency of the binding conditions is higher with each cycle to isolate binders with higher affinity. The purity of cell pool is getting higher and majority of the cells should be specific antigen-staining positive after 2-4 cycles of enrichment.

This repetition of screen not only enriches the binder but shuffles the heavy and light chain in cells, which may increase the chance to insolate a antibody with higher expression as well as higher affinity.

For analysis of the cell polls with FACS, the cells are stained with FITC-conjugated anti-light chain antibody or PE-conjugated specific antigen or both.

The primary cell pool and sub-cell pools are FACS analyzed to calculate enrichment efficiency.

To isolate the antibody genes, the total mRNA is extracted from the final sub-cell pool. The Vh and Vl regions are RT-PCR amplified, restriction enzyme digested and purified.

Obtained Ch and Cl are inserted into stable expression vector from example 2 in frame with its constant partner to form a full-length heavy chain gene and full-length light chain gene to build a stable expression sub-library. In this vector the pair of heavy and light chain is fixed and only one type of antibody is expressed if only one vector is integrated in the genome.

The stable expression sub-library is transfected into CHO cells. To ensure the single vector integration into each cell genome, the transfection conditions are optimized in multiple parameters including DNA dose, mixing with different amount of non-expression vector, and transfection procedure.

The expression level and specific binding affinity of the antibodies are analyzed by FACS. The single cells which exhibit high expression and high affinity are isolated by single cell FACS sorting.

After expansion, theses single cell populations are further analyzed by:

1. Antigen competition analysis on cell surface.

2. Cross-reaction of the antibody with antigen of other species.

3. Function analysis using condition medium.

4. Function analysis using purified antibody.

The specific antibody genes are cloned by RT-PCR from these single cell populations and sequence confirmed that they are single clones. The selected antibody gene can be expressed in large scale for further analysis.

### Example 5

This example describes construction of full-length mouse-human chimerical antibody mammalian display library

To build the library, the same strategy as described in Example 3 is used.

The genes of variable domain of mouse antibody heavy chain and light chain (Vh and Vl) are amplified by PCR from ready-to-use cDNA of mouse immune tissue (bone marrow, spleen and peripheral blood lymphocytes) (BioChain, South San Francisco, Calif. USA), using primers as described in book Phage Display (Carles et al, Cold Spring Harbor Laboratory Pr.). The PCR conditions are optimized to ensure the efficient and accurate amplification of Vh and Vl. The PCR products are digested with suitable restriction enzymes matched with cloning vector. After digestion the products are purified using PCR purification kit (Qiagen).

For mouse-human chimerical heavy chain expression, the human heavy chain constant region (Ch) of IgG1, FCS and PDGFR sequence are cloned into the vector from example 1 in MCS. Then the mouse Vh mixture is inserted into the vector in frame before Ch region using T4 ligase. After transformation of ligation mixture, the transformation efficiency and library size are calculated by measuring the transformant number to make sure the library size is 10e5 or larger. The diversity of library is analyzed and calculated more accurately by sequence analysis of more than 100 individual clones. The FCS region may not be included in the vector if the antibody is only need to be expressed on cell surface.

For mouse-human chimerical light chain expression, the human kappa (or lambda) chain constant region (Cl) is cloned into the vector from example 1 in MCS. Then the mouse Vl mixture is inserted into the vector in frame before Cl region using T4 ligase. After transformation of ligation mixture, the transformation efficiency and library size are calculated by measuring the transformant number to make sure the library size is 10e4 or larger. The diversity of library will be analyzed and calculated more accurately by sequence analysis of more than 100 individual clones.

Both heavy chain and light chain library vectors can be easily amplified in bacteria and stored in −20 C with safe.

This library is a primary, naïve full-length mouse-human chimerical antibody library with a potential diversity of >=10e9. The antibodies expressed from this library can be anchored on mammalian cell surface for affinity and function screening and selecting directly. At the same time, part of the antibodies are expressed as soluble molecules into condition medium, which can be used directly for further function analysis and characteristic analysis of antibodies without the conversion of antibodies from membrane anchored format to soluble format, speeding up the screening and selecting.

To construct a full-length mouse antibody display library, the mouse constant regions of heavy and light chains are used to replace the human counterparts in the vector.

### Example 6

This example describes construction of antibody-like bivalent peptibody mammalian display library

Using the method as described in above examples, an antibody-like, bivalent, mammalian displayable peptibody library is constructed. A polypeptide gene library is in vitro synthesized. The DNA sequence of the library has a size of 36-72 nucleotides franked at both ends with restriction enzyme recognizing sequences for cloning the library into expression vector.

Two different peptibody libraries are constructed. In one library, the polypeptide is fused to human Ch and in the other, fused to human Cl. Both libraries are with a size of =>10e5.

These two DNA libraries are mixed with equal amount of molecules and transfected into mammalian cells (CHO cells). Because of the ex vivo paring in cells, the potential library size could reach to 10e5 or larger. The specific binders of selected antigen are screened out by the methods as described in Example 4. In this antibody-like peptibody, there may be possible up to 4 different polypeptides, two from heavy chain fusion and two from light chain fusion. Further screen and down stream development such as pairing in a vector with dual expression cassette can finally identify a candidate with pair of polypeptides, having desired function.

### Example 7

This example shows the construction of a single expression cassette vector for rapid construction of antibody libraries and expression of antibody proteins on mammalian cell surface.

### Design and Construction of Expression Vector

Restriction enzymes SfiI recognizes a non-palendromic sequence and forms a 3-base protruding end. Because each of these three positions at end can be any one of four nucleotides G, A, T, C, the sequences of the ends can have 64 different possibilities in total. So it is possible to design different cutting sequences at both ends of fragments which will not self-ligate during ligation. This feature is very useful in library construction to increase the ligation and transformation efficiency. BstXI and BsmBI have similar feature and each can form 256 possible ends. For flexible insertion of antibody heavy chain, light chain or variable domain of heavy chain, one universal vector has been designed and above listed three enzymes have been introduced into the vector. CMV promoters have been chosen to drive the expression of inserted genes in this vector.

TABLE 1
Primers for vector construction

| Names | Sequences |
|---|---|
| P1-172 | TGGCCACATAGGCCGTCTCTAGTCCACCATGGACTGGACCTG |
| | GAGGATC |
| P2-087 | GTCCACCTTGGTGTTGCTGGGCTT |
| P3-101 | AAGCTGGCTAGCCACCTGATTGGCCACATAGGCCGTCTCTAG |
| | TCCACCACCATG |
| P4-145 | CCCTTGGTGGAGGCTGAGGAGACGGTGACC |
| P5-144 | GGTCACCGTCTCCTCAGCCTCCACCAAGGG |
| P6-142 | GCGTGTCCTGGCCCACAGCATTGGATCCTTTACCCGGAGACA |
| | GAGAGAGG |
| P7-143 | GCCTCTCTCTGTCTCCGGGTAAAGGATCCAATGCTGTGGGCC |
| | AGGACACGC |
| P8-104 | TGGCCGTGCAGGCCTTATCAACGTGGCTTCTTCTGCC |
| P9-103 | GTCGACCTCGAGCCAGAGTCATGGCCGTGCAGGCCTTATCA |

The vector constructed contains full length human heavy chain fused at the 3′ end with trans-membrane domain (TM) of platelet derived growth factor receptor (PDGFR), To reduce the size, the vector we designed contains no selection gene expression cassette, which usually would be used for the selection in mammalian cells. To obtain a full length human antibody heavy chain fused with PDGFR-TM (HC-TM) and introduce listed enzyme cutting sites into the vector, 9 primers have been designed (Table 1). HC-TM was obtained by 5 PCRs. Using total RNA isolated from human PBMC as a template, variable domain of heavy chain has been first RT-PCR amplified by primer P1 and P2 (PCR-1). Then PCR-2 was performed using primers P3 and P4 and the products from PCR-1 as template to introduce NheI/BstxI/SfiI/BsmBI enzyme recognizing sequences before the start codon and the second BsmBI recognizing sequence in frame at the 3′ end of the variable domain. Human IgG-1 constant region was RT-PCR amplified from total RNA isolated from human PBMC by P5 and P6 (PCR-3) to introducing second BsmBI recognizing sequence at 5′ end and BamHI recognizing sequence at the 3′ end. The 4^{th} RT-PCR was performed using P7 and P8 to amplify PDGFR-TM. At the 5′ end of TM, a BamHI cutting site was incorporated. The final PCR (PCR-5) used fragments from PCR 2, 3, and 4 as templates and primers P3 and P9 to introduce SfiI/BstXI/XhoI recognizing sequence at the 3′ end of this fusion protein. The final PCR product (fragment 5) contains full length human heavy chain and PDGFR-TM. After digestion with NheI and XhoI, the fragment was inserted into the vector pcDNA 5/FRT between NheI and XhoI at multiple cloning site to form the interim vector.

To reduce the total size of the vector, the hygromycin B selection gene expression cassette was deleted from the interim vector. In that way, same amount of vector DNA used in transfection will contain more copies of plasmid molecule, hopefully, to increase the diversity being screened every time. Therefore primers P201 (5′-ctaactgacacacattccacagaagcttcaccctaatcaagttttttgggg-3′) and P202 (5′-tgtatcttatcatgtctgtataccgaagcttcctctagctagagcttggcg-3′) were designed to contain HindIII recognizing sequence and complimentary to the interim vector. Using these two primers, a 5.2 kb fragment was PCR-amplified from interim vector, which contains all the components for the vector's replication in bacteria and the antibody gene expression in mammalian cells. After digestion with HindIII, the fragment was circularized by self ligation to form the final expression vector pDGB-HC-TM (FIG. 3).

### Enzyme Digestion Confirmation of the Vector and Analysis of Ligation and Transformation Efficiency

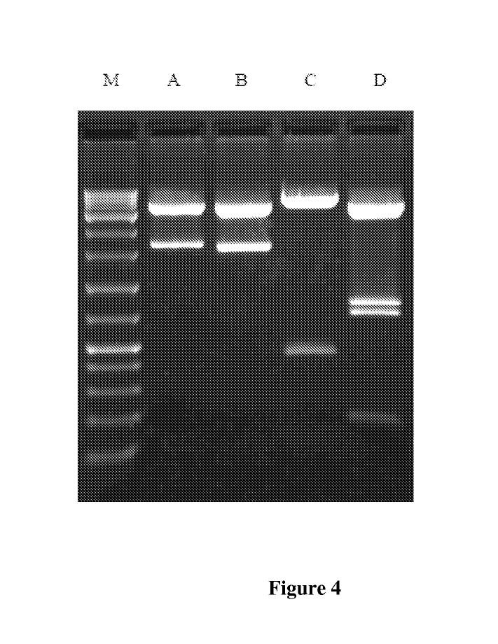

To confirm that the pDGB-HC-TM contains all proper restriction enzyme recognizing sequences and can be efficiently digested to form right sized fragments, maxi-prep vector DNA has been analyzed by proper enzyme digestion. Results (FIG. 4) show that digestion of the vector by five different enzymes in four combinations gives expected DNA fragments. This result indicates that the vector pDGB-HC-TM contains all introduced enzyme cleavage sites at the right location. To test that all these fragments can be re-ligated to form proper vector, all these fragments have been individually purified. Ligation was performed in four groups as indicated in FIG. 4. The vector fragment and insert fragment were mixed in a ratio of 1:1 in each group. After transformation, the efficiency has been calculated. Results show that using competent cells with an efficiency of 3×10⁷, the ligation-transformation efficiency of 1 ug of DNA can reach up to 3.2×10⁶ (Table 2). The transformation efficiency of group 1, where the vector was digested by NheI and XhoI, has an efficiency of 0.94×10⁶, lower than the other three groups. The vector fragment from NheI and XhoI digestion can be self-ligated without insert fragment. The vector fragments from other three groups show no self-ligation. It is reasonable to say that this vector self-ligation reduces the effective ligation between vector fragment and insert fragment in real ligation, decreasing the transformation efficiency and increasing the background (8.6%, Table 2-A). To confirm that the fragments are correctly ligated to form the full length vector, 4 colonies were randomly picked from each group for analysis and all 16 clones (4 for each ligation) show right sized fragments.

TABLE 2
Table 2: Analysis of ligation and transformation efficiency *

| | V + I ** | V ** | V only/ | Colony #/ |
|---|---|---|---|---|
| | 10 μl | 100 μl | V + I | μg DNA ** |
| A (NheI + XhoI) | 329 | 284 | 8.6% | 937650 |
| B (SfiI) | 576 | 250 | 4.3% | 1641600 |
| C (BsmBI) | 1127 | 323 | 2.9% | 3211950 |
| D (BstXI) | 572 | 66 | 1.2% | 1630200 |

* Total volume of transformation culture is 285 μl.
** V + I: Vector fragment + Insert fragment; V: Vector fragment only.

### FACS Analysis of Antibody Expression on Cell Surface

The vector pDGB-HC-TM contains only HC-TM fusion protein but full length antibodies need simultaneous expression of heavy chain and light chain in single cells. Using primer P182 (5′-AGCCACCTGATTGGCCACATAGGCCTGAACCACCATGGTGTTGCAGACCCAGGTC-3′) and P108 (5′-ccagagtcatggccgtgcaggccTTATCAAGACTCTCCCCTGTTGAAGCTC-3′), full length human kappa chain gene was amplified by two-step RT-PCR. After SfiI digestion, the gene was inserted into the vector between SfiI cutting sites to replace the HC-TM in pDGB-HC-TM to form the human kappa chain expression vector pDGB-huKappa. The sequence of kappa gene was confirmed by sequencing analysis. The vectors pDGB-HC-TM and pDGB-huKappa were co-transfected into 293-T Cells. The transient expression of antibodies on cell surface was analyzed by FACS 60-hours post transfection. Parental 293-T cells show no antibody expression on cell surface when staining with PE-conjugated mouse anti-human kappa chain antibodies and FITC-conjugated mouse anti-human IgG antibodies. The cells transfected by pDGB-HC-TM show heavy chain expression on the cell surface but no detection of kappa chain expression. Cells transfected by pDGB-huKappa, neither heavy chain nor light chain were detected on cell surface. When the cells were transfected by co-transfection of pDGB-HC-TM and pDGB-huKappa, the heavy chain and light chain can be separately and simultaneously detected. These results suggest that if the light chain can be detected on the cell surface, the cell will contain heavy chain too. Only heavy chain and light chain co-expressed in the cells, the full length antibodies can be detected on the cell surface.

To see whether this vector can be used to construct antibody library effectively, we have used two-step RT-PCR to amplify full length kappa chain library (P182 and P108) and variable domain of heavy chain (Vh) library (P149, 5′-TGGCCACATAGGCCGTCTCTAGTCCACCATGGACTGGACCTGGAGGATC-3′ and P150, 5′-GCCCTTGGTGGAGGCTGAGGAGACGGTGACCAGGGTGCC-3′) and separately inserted both libraries into the vector. After co-transfection of these two libraries into 293-T cells, the antibody expression was analyzed by FACS. Both light chain and heavy chain can be detected on cell surface, suggesting that this vector can not only be used in cloning of single genes but also for libraries.

### Discussion

Antibody libraries were constructed by insertion of antibody genes into a proper vector to form a recombinant plasmid DNA. After introduced into a proper host cell, the antibody will be expressed for screening and selection. One of the key steps in construction of antibody libraries is effective ligation of vector fragment and insert fragment. Commonly used restriction enzymes form palendromic ends after digestion, allowing self-ligation occur and reducing the ligation efficiency. Scientists commonly use two different methods to prevent from self-ligation of fragment. First way is using two different enzymes to digest the target DNA. The single copy of vector fragment or insert fragment will not self-ligate but the multiple vector fragments or insert fragments can still ligate. This method cannot prevent self-ligation of vector or insert. Another way is dephosphorization of the vector fragment. Because only one fragment was dephosphorized, another fragment, no matter insert fragment or vector fragment, can still self-ligate. So second method cannot completely prevent self ligation either. Here we reported the use of three restriction enzymes which recognize and cleave to form non-palendromic ends. Theoretically, SfiI can recognize and cleave the target DNA to form 64 different ends. BsmBI and BstXI can form 265 different ends. This feature makes it easier to design two recognizing and cutting ends which can be properly enzyme digested but not allow for self-ligation. Table 3 listed 6 sequences recognized by three enzymes used in the vector. The results demonstrated that these ends do not self-ligate and only matched two ends can stick together to be ligated, dramatically increased the ligation and transformation efficiency in the library construction. Using competent cells with an efficiency of 3×10⁷, the transformation efficiency can reach up to 3×10⁶ per microgram of ligated DNA. Currently, competent cells with transformation efficiency up to 10⁹ are commercially available. It is reasonable to say that the transformation efficiency of the ligation mixture from this experiment could be much higher if using these high transfection efficiency competent cells. It is worth to mention that the background of ligation in this report was 1.2% to 4.3%, much lower than the group using NheI and XhoI (8.6%). This will not only increase the library quantity but also library quality. To be noted that, in group 4, there are two extra BstXI sites present in cloned genes and the ligation is actually 4-way ligation. Fortunately, sequence analysis indicates that all of these four pairs of ends created by BstXI digestion will not form self-ligation, which has been proved by high transformation efficiency and low background with BstXI digestion group (Table 2).

TABLE 3
Nucleotide sequences in pDGB-HC-TM recognized
by three restriction enzymes

| Names | Recognizing sequences | |
|---|---|---|
| BsmBI | 5′-C G T C T C T | A G T C C-3′ |
| 1 | 3′-G C A G A G A T C A G | G-5′ |
| BsmBI | 5′-C G T C T C C | T C A G C-3′ |
| 2 | 3′-G C A G A G G A G T C | G-5′ |
| SfiI | 5′-G G C C A C A T | A G G C C-3′ |
| 1 | 3′-C C G G T | G T A T C C G G-5′ |
| SfiI | 5′-G G C C T G C A | C G G C C-3′ |
| 2 | 3′-C C G G A | C G T G C C G G-5′ |
| BstXI | 5′-C C A C C T G A | T T G G-3′ |
| 1 | 3′-G G T G | G A C T A A C C-5′ |
| BstXI | 5′-C C A T G A C T | C T G G-3′ |
| 2 | 3′-G G T A | C T G A G A C C-5′ |

The vector pDGB-HC-TM contains IgG1 heavy chain constant region and transmembrane domain. Using BsmBI cutting sites, a Vh library can be easily inserted into the vector. Using BstXI or SfiI, the HC-TM can be replaced. Using BamHI cutting site between CH and TM and downstream XhoI site, the TM can be easily deleted to express soluble antibodies. These features make this vector universal for the construction of antibody libraries of full length heavy chain, full length light chain, or variable domain of heavy chain.

Heavy chain and light chain can be clearly detected on the cell surface when both genes were co-transfected into cells. It is worth to mention that when only heavy chain expression vector was introduced into cells, heavy chain can still be detected on the cell surface without the present of light chain. This result suggests that in transient transfection, heavy chain can individually be displayed on the cell surface. When transfected only by light chain expression vector, there are no light chains being detected on the cell surface. It was known from the co-transfection that the light chain should have been expressed but just not displayed on the cell surface because there is no TM fused to the end of light chain. Therefore, detection of the light chain on the cell surface means the presence of heavy chain on the cell surface and using anti-light chain antibodies can quantitatively analyze full length antibodies on the cell surface. Using transient transfection and low concentration of fluorescence conjugated specific antibodies ( 1/7^{th} to ¼^{th} of manufacture's suggested dosage), 50 to 60% of the cells show antibody expression on cell surface. This result suggests that the vector constructed can efficiently express carried antibody genes.

We have further investigated the application of the vector in construction of antibody library by inserting PCR amplified Vh library and full length kappa chain library into the vector separately to establish a full length heavy chain library and full length light chain library. After co-transfection of these two libraries into 293 T-cells, FACS can detect 40% of cells displaying antibodies. Not every gene inserted in the vector has correct reading frame and can be expressed efficiently. 40% of positive cells should represent the effective expression of correct antibody genes in the library. Considering that only 60% of the cells are positive when co-transfected with sequence-confirmed single heavy chain and single light chain, 40% of positive rate indicates that the expressible antibody genes in each library could be in a range of 60-80%.

In conclusion, we have constructed a universal mammalian expression vector which can be used to construct an antibody library rapidly and to express contained antibody genes with high efficiently.

### Example 8

### Design, Construction, and Analysis of a One-Step Dual-Expression Cassette Vector

Example 7 has shown successful construction of a universal single expression cassette vector utilizing restriction enzymes BstXI, BsmBI and SfiI, which recognize and cleavage unique sequences to prevent self ligation, and demonstrates high insertion efficiency of HC or LC. By using a 1:1 molecule ratio of vector and insert without further optimization, the ligation and transformation efficiency of this single expression cassette vector can easily reach 10⁷ clones per μg of ligated DNA. However, this vector is only capable of single heavy chain or light chain insertion. Based on the structure of the vector, we designed a new vector, pDGB4 for simultaneous insertion of both HC (or VH) and LC through four-way ligation and pDGB4-FCS for simultaneous expression of antibodies in both membrane-anchored form and soluble form as illustrated in FIG. 5A.

To construct the vector, 8 primers (Table 4) were synthesized to introduce the proper restriction enzyme-recognizing sequences into the vector at the desired location and to generate four fragments after PCR-amplification—P1 and P2 for 5.0 kb frame fragment, P3 and P4 for 1.6 kb HC-TM (heavy chain fused to transmembrane domain) fragment, P5 and P6 for 1.8 kb frame fragment and P7 and P8 for 0.7 kb light chain fragment. After digestion of the fragments with proper restriction enzymes and purification, four fragments were mixed in a molar ratio of 1:1:1:1 with a total amount of 91 ng in 10 μl of total ligation mixture. One μl of ligation mixture was then used in the transformation. Out of 265 colonies, 16 colonies were analyzed by digestion with enzyme BstXI, and 9 out of 16 showed the right-sized fragments. To confirm that the constructed vector contained all of the desired restriction enzyme cutting sites and can express both heavy and light chains, clone 16 was chosen for further analysis.

TABLE 4
Primers used in construction of pDGB4

| Primer | | |
|---|---|---|
| number | Primer Name | Primer Sequence (5′---- 3′) |
| P1 | 5.0 kb-F | AGTCTTGATAAGGCCTGCACGGCCCTCGAGTCTAGAGGGCCCG |
| P2 | 5.0 kb-R | ACTGAGACGCCAATCAGGTGGCTAGCCAGCTTGGGTCTCC |
| P3 | 1.6 kb-F | AAGCTGGCTAGCCACCTGATTGGCGTCTCTAGTCCACCATGGAC |
| P4 | 1.6 kb-R | TTCCCATGGTCCAGAGTCATGGTTATCAACGTGGCTTCTTCTGCC |
| P5 | 1.8 kb-F | ACGTTGATAACCATGACTCTGGACCATGGGAAATGTCAGAGTGGAG |
| P6 | 1.8 kb-R | TGGTGGTTCAGGCCTATGTGGCCTAGCCAGCTTGGGTCTCCC |
| P7 | 0.7 kb-F | AAGCTGGCTAGGCCACATAGGCCTGAACCACCATGGTGTTGCAG |
| P8 | 0.7 kb-R | TAGACTCGAGGGCCGTGCAGGCCTTATCAAGACTCTCCCCTGTTG |

Maxiprep DNA of clone 16 was digested in five different ways (FIG. 5B). Results show that all 5 different digestions give the right-sized fragments, suggesting that the vector contained all the designed enzyme cutting sites at the correct locations. However, the fragments are from PCR amplification and do not guarantee expression; therefore, to confirm that the heavy chain and light chain contained in the vector can be expressed appropriately, DNA was transiently transfected into 293-T cells, and the expression of both heavy and light chain was analyzed by FACS. Compared to the control, both heavy chain and light chain can be separately and simultaneously detected. These results demonstrate that our vector pDGB4 can successfully express full-length antibodies on the cell surface.

### Four-Way Ligation Efficiency Analysis

The purpose of the construction of this vector is to facilitate the simultaneous and efficient cloning of heavy chains and light chains. To analyze how high the ligation and transformation efficiency can be, the vector DNA of clone 16 was digested by BstXI plus SfiI (FIG. 5B lane 4) and BsmBI plus SfiI (FIG. 5B lane 5), and the fragments were individually purified after electrophoresis separation. Two four-way ligations and transformations were carried out, and ligation and transformation efficiency was calculated

TABLE 5
Cloning efficiency of 4-way ligation

| | | | V only/ | Colonies/ |
|---|---|---|---|---|
| | Number of colonies per plate | | V + I | μg of |
| Enzymes | Vector and insert | Vector only | Ratio | DNA |
| “1” BsmBI and SfiI | 233 | 16 | 2.70% | 259000 |
| “2” BstXI and SfiI | 754 | 17 | 0.90% | 838000 |

Note:
i) The final volume of transformation mixture is 500 μl. 50 μl were plated for later colony analysis. The transformation efficiency of positive control pUC19 is 107000/ng DNA. The negative control is DH5a competent cells without vector transformation, and the colony number is 0 (not shown).
ii) One μl of ligation mixture (9 ng of total DNA) was used for each transformation.

To confirm that the clones used to calculate the efficiency actually contained the right vectors, 16 colonies were selected (8 from ligation one and 8 from ligation two) for analysis. Plasmid DNAs of 16 clones were digested by proper restriction enzymes and all 16 clones contain right-sized fragments, further supporting the significantly high efficiency of our four-way ligation. Results also show that the V-only backgrounds were in a range of 0.9-3.6%. This undeniably proves that our method of four-way ligation ensures both high quantity and high quality (Table 5).

To mimic the realistic conditions in pharmaceutical library construction, PCR-amplified human kappa chain library and human heavy chain variable domain library were used in the four-way ligation to replace the gene fragments obtained from digestion of clone 16. The ligation and transformation efficiency is 1×10⁵ per μg of ligated DNA.

### Insertion and Sequence Confirmation of Furin Cleavage Site (FCS) in the Vector

Using vector pDGB4, we can introduce both HC and LC into a cell by transfection of a single vector to express full length antibodies on the cell surface. Though membrane-bound antibodies are useful for FACS analysis and antibody selection, secreted antibodies are necessary for actual function assays. It will be ideal if the antibody can be expressed in both membrane anchored form and soluble form simultaneously. Furin is a cellular protease which recognizes a consensus amino acid sequence, RXRR, and cuts proteins which contain this sequence right after the fourth R when they reach the transgolgi network. Insertion of furin cleavage sequence in a gene of virus membrane protein can express the protein in soluble form. Because furin cleaves partially, insertion of furin cleavage site (FCS) into pDGB4 between the heavy chain constant domain (Ch) and PDGFR trans-membrane domain (TM) may implies that a portion of the antibodies will be released into culture medium while the other portion of the antibodies will remain attached to the cell membrane.

The FCS sequence was inserted into the vector pDGB4 to form the vector pDGB4-FCS and clone #3 was sequencing confirmed to contain the FCS in frame between HC and TM (FIG. 5A).

### Analysis of Antibody Expression Through FCS-Containing Vector

To compare the antibody expression on the cell surface with and without FCS, the Flip-In system was used (Invitrogen). Flip-in system comprises of vector pcDNA™ 5/FRT, vector pOG44, Flip-in Chinese hamster ovary (FCHO) cell line, and related cell maintenance medium. In Flp-In system, only a single copy of transfected vector will be integrated into one cell genome in a designated location through recombinase-mediated DNA recombination. In that way, the expression of same antibody from different vectors will only be dependent on that vector's characteristics, and we would not have to consider underlying variables such as copy numbers of the integrated vector, or the location of integration.

Plasmid DNAs of vector pDGB4-FCS, clone #3, as well as pDGB4, #16 and #78, was stably transfected into FCHO cells by co-transfection with pOG44 which contains a recombinase gene. All three clones contain same antibody gene. Clone #3 contains both TM and FCS regions; clone #16 contains TM but no FCS sequence, and all of the antibodies expressed will be membrane-anchored. Clone #78 contains neither TM no FCS at the end of HC, and all of the antibodies expressed will be secreted into the culture medium. Therefore, the amount and ratio of antibodies on the cell surface and in conditioned medium will only be dependent on the function of FCS and TM.

Three selected cell pools from clone #3, #16 and #78 were stained by PE-conjugated mouse anti-human kappa chain antibody and FACS analyzed. As expected, 90% of cells from #16 are detected to express antibodies on the cell surface and no antibody expression was detected on the cell surface of #78. In contrast to clone #16 and #78, 30% of cells from #3 have antibodies expressed on the cell surface, indicating that some of the antibodies may have been cleaved and secreted into the conditioned medium.

To confirm this inference, Western blotting was performed. The results show that no antibodies were detected in the condition media from FCHO and clone #16. Compared to the signal from clone #78, the signal from clone #3 was much weaker. The estimated concentration in conditioned media is 1-2 ng per μl for #3 and 4-5 ng per μl for #78. This is in full concurrence with the results from our FACS analysis. These results prove that insertion of FCS has successfully and efficiently manipulated the dual cassette vector so that it can express antibodies in both membrane-bound form for FACS analysis and secreted form for function assay.

### Discussion

The future of antibody library construction and pharmaceutical applications of antibodies depends largely on screening and selecting platforms. Although past display technologies have been effectively used in a wide variety of applications, the quantity and quality of the libraries constructed have not yet met current needs for more efficient development of antibody drugs. As the need for therapeutic antibodies for treatment of human diseases increase, platforms will transition more towards mammalian cell display and require advancements in all aspects of library construction. Our vector provides a step to this transition by combining the major benefits of past display techniques and overcoming their shortcomings. Because our platform displays full-length antibody on mammalian cells, it contains inherent advantages over phage and yeast displays. Furthermore, since our platform is a vector system in human mammalian cells, it also surpasses hybridoma technology which relies on animals.

Instead of the classic enzymes such as HindIII or BamHI, we have used three different restriction enzymes in our vector: BstXI, BsmBI, and SfiI. Utilization of these enzymes is the key to the increase in the ligation efficiency of our vector and to the insertion of both heavy and light chains in only one step. Because the enzymes were designed to cleave non-palendromic sequences, all manners of self-ligation become impossible. Each individual border of every single vector fragment is unique and can only bind to its complimentary border during ligation. Since we can control which borders will bind, there is no need to worry about self-ligation as a hindering variable. Therefore, we can ligate all four fragments in the vector at once. To the best of our knowledge, this revolutionary use of a one-step four-way ligation for library construction has not been reported so far. Our results have demonstrated that the efficiency of this method truly exceeds other display platforms' reports. The use of our one-step vector system and removal of self-ligation not only reduces time consumption during library construction, but also increases the overall quality of the antibody library.

In addition to reducing time consumption and increasing efficiency in library construction, our display system can generate both membrane-bound antibodies for screening and soluble antibodies for function assay. The human antibody on cell surface can be detected by very low concentration of PE-conjugated anti-human kappa chain antibody (one seventh of manufacture's suggested dose) and the concentration of human antibody in conditioned medium was estimated at 1-2 ug per ml. Therefore, the insertion of FCS into vector pDGB4 allows for the selection of high affinity antibodies through FACS and direct use of culture medium from the cells for function assays. The use of cleavage activity of inherent furin in combination with anchoring ability of transmembrane domain to express secreted and membrane-bound antibodies simultaneously has not been reported before.

Because the antibody genes were inserted through enzyme digestion, any other genes can also be inserted as long as they have the correct ends. Thus, future applications of our vector platform may easily exceed antibody expression and become pivotal in any protein expression experiments. For example, it can be used to clone other pairs of genes such as receptor and ligand or both subunits of T-cell receptors. Co-expression of these molecules through single vector could even help in quantitatively analyzing the relationship between the molecules.

In conclusion, we have developed a novel full-length antibody mammalian display system. The unique characteristics of this system include rapid construction of full-length human antibody display libraries by four-way ligation and simultaneous expression of antibodies in both membrane-bound form and soluble form for functional screening and selecting of full length monoclonal antibodies with high expression and affinity.

### Example 9

This example shows the construction of a full-length human antibody mammalian display library with combinatory diversity more than 10¹¹.

10⁹ of peripheral blood mononuclear cells (PBMC) were isolated from more than eighty donors. Total RNA was isolated from PBMC using RNA Easy kit (Qiagen). The genes of variable domain of human IgG1 heavy chain (Vh) and kappa light chain (LC) were amplified by two-step RT-PCR. The PCR products were digested with suitable restriction enzymes matched with cloning vector. After digestion, the products were purified by gel extraction kit (Axygen, Union City, Calif.).

The Vh library was inserted into the vector of example 1 in frame before human immunoglobulin constant region (Ch) using T4 DNA ligase. After transformation of ligation mixture, the transformation efficiency and library size were calculated by counting the number of transformants and the size of human IgG1 heavy chain library was 1.32×10⁶. Vh regions of 10 colonies were sequence analyzed. 8 out of 10 had right coding sequences.

Full-length kappa chain library was cloned into the vector of example 1. The library size was 6.21×10⁵. Kappa genes of 10 colonies were sequence analyzed. 8 out of 10 had right coding sequences.

To measure what percentage of the library were expressible, the plasmid DNA of ten heavy chain clones were co-transfected with single kappa chain clone which had been tested to express kappa chain in high level and the plasmid DNA of ten kappa chain clones were co-transfected with single heavy chain clone which had also been tested to express heavy chain in high level. The expression of antibody on transfected cell surface was FACS analyzed. 6 out of 10 heavy chain clones and 5 out of 10 kappa chain clones were expressed in different levels.

The combinatory diversity of the libraries was 8.2×10¹¹ theoretically, 5.25×10¹¹ in DNA level and 2.46×10¹¹ in expression level respectively.

This library was a primary, universal full-length human antibody (immunoglobulin G1/Kappa) library. The antibodies expressed from this library were anchored on mammalian cell surface for affinity and function screening and selecting directly.

Using same strategy, several specific human antibody display libraries were constructed with diversities between 10⁹ and 10¹¹. The libraries included human HBV specific, human renal cancer specific library, human self-immune disease specific libraries, and personal specific libraries.

Using same strategy, a human tumor specific, mouse-human chimerical antibody (Immunoglobulin G1/kappa) mammalian display library was constructed by using RNA isolated from spleen cells of Kuanming mice immunized with human tumor cells (Liver cancer 7721, lung cancer A549 and Colon cancer DLD-1). The library had a combinatory diversity of 1.11×10¹⁰ theoretically and 1.78×10⁹ in DNA level.

Using same strategy, two more chimerical antibody display libraries were also constructed. One was non-immunized mouse-human chimerical antibody library constructed by using RNA isolated from normal Kuanming mice spleen and the size of combinatory library was 7.99×10¹⁰. The other was non-immunized rabbit-human chimerical antibody library constructed by using RNA from normal xingxilang rabbit and the size of combinatory library was 7.57×10¹⁰.

### Example 10

This example describes the construction of antibody-like bivalent peptibody (ALBP) mammalian display library

The peptide libraries were constructed using assembling PCR with mouse signal peptide in frame at 5′-end of the genes and proper restriction enzyme cutting sequences franked at both ends. The DNA sequence of the peptide library had a size of 36 nucleotides, coding for a peptide of 12 amino acids.

Two different peptibody libraries were constructed. In one library, the polypeptide was fused to human IgG1 constant region (PepG library) and in the other, fused to constant region of human kappa chain (PepK library). PepG and PepK libraries had sizes of 3.96×10⁵ and 1.19×10⁶ respectively. 10 pepG clones and 9 pepK clone were sequence analyzed. 7 out of 10 pepG gave sequence results and 4 out of 7 had right coding sequences. 6 out of 10 pepK clones had right coding sequences.

The combinatory diversity of the library was 4.7×10″ theoretically and 1.6×10″ in DNA level respectively.

FACS analysis was carried out using FITC-conjugated TNF, HBsAg and proteins from human colon tumor DLD-1 cells and binding of these antigens to the ALBP library-transfected 293-T cells was demonstrated.

### Example 11

This example shows the construction of HIV-specific human antibody library and isolation of HIV gp120 specific antibodies from the library

PBMC was isolated from HIV-infected long survivor donor and total RNA was isolated from PBMC using RNA Easy kit (Qiagen). The genes of variable domain of human immunoglobulin heavy chain (Vh) were amplified by two-step RT-PCR. The amplification of human kappa chain was not successful. PCR products of Vh were digested with suitable restriction enzymes matched with cloning vector. After digestion the products were purified by gel extraction kit (Axygen, Union City, Calif.).

The Vh library was inserted into the vector of example 1 in frame before IgG1 constant region (Ch) using T4 DNA ligase and the size of heavy chain library was 1.16×10⁵ (HIV-Vh-Lib).

The HIV Vh library was also inserted into the vector of example 2 by 4-way ligation. In the construction, the single kappa chain gene CZR1 was used instead of kappa chain library. The library size was 7.8×10⁴ (HIV-CZR-Lib).

To analyze the expression of antibodies, the HIV-CZR-Lib was transfected into FCHO cells and selected under hygromycin (500 ug/ml). The stable selected cell pool was stained by FITC-conjugated HIV envelope protein gp120 (FITC-gp120) and analyzed by FACS. About 3% of the cells were shown the specific antigen binding, indicating the presence of gp120-specific antibody on cell surface. This portion of the cells was isolated by FACS and the Vh genes coding for the specific antibodies were PCR amplified and analyzed by standard molecular biology techniques. One of the clones showed specific binding to FITC-gp 120.

### Example 12

This example shows the composition of human antibody library construction kit and the rapid construction of a personalized human antibody library by this kit.

The core kit contained: 1) single expression cassette vector; 2) dual expression cassette vector; 3) human heavy chain primer set 1, 2 and 3 (HGP1, HGP2 and KGP3) and 4) human kappa chain primer set 1, 2 and 3 (HKP1, HKP2 and HKP3).

6 ml of peripheral blood were obtained from a single donor and 3×10⁶ PBMC were isolated from the blood. 1500 ng of total RNA were purified from the PBMC and the genes of Vh and kappa chain were RT-PCR amplified.

The amplified antibody genes were purified, digested by proper restriction enzymes and inserted into the single expression cassette vector. The sizes of heavy chain and kappa chain libraries were 9.4×10⁴ and 8.4×10⁴ respectively. The size of combinatory library was 7.9×10⁹.

FACS analysis of 293T cells that were co-transfected by heavy and light chain libraries demonstrated the expression of antibodies on cell surfaces.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of isolating a polynucleotide encoding an antibody specifically recognizing a specific antigen, comprising:
a) screening an initial population of mammalian cells transiently transfected with a first primary library of polynucleotides encoding light chain and a second primary library encoding heavy chain and displaying antibodies encoded by the polynucleotides on the cell surface to obtain a subpopulation of mammalian cells;
b) reverse transcribing mRNA extracted from said subpopulation of mammalian cells into cDNA and amplifying said cDNA to generate a sublibrary of polynucleotides;
c) screening a population of mammalian cells transfected with the sublibrary of polynucleotides and displaying antibodies encoded by the polynucleotides on the cell surface to obtain a second subpopulation of mammalian cells; and
d) isolating from said second subpopulation of mammalian cells a polynucleotide encoding an antibody specifically recognizing a specific antigen.

2. The method of claim 1, wherein steps b) and c) are repeated at least once prior to step d).

3. The method of claim 2, wherein steps b) and c) are repeated no more than about three times prior to step d).

4. The method of claim 1, wherein the polynucleotides in the primary libraries encode at least 10⁹ different antibodies.

5. The method of claim 1, wherein the initial population of mammalian cells are transiently transfected with the primary libraries under a condition where an individual mammalian cells in the population can take up more than about 1000 copies of the polynucleotides.

6. The method of claim 1, further comprising transiently transfecting the primary libraries of polynucleotides into the initial population of mammalian cells.

7. The method of claim 1, wherein step a) comprises:
(i) contacting the initial population of mammalian cells with the antigen under a suitable binding condition; and
(ii) recovering a subpopulation of mammalian cells that bind to the antigen.

8. The method of claim 7, wherein the recovery is carried out by FACS, magnetic beads, or a combination thereof.

9. The method of claim 1, wherein step c) comprises:
(i) contacting the enriched mammalian display library with the antigen under a suitable binding condition; and
(ii) recovering a subpopulation of mammalian cells that bind to the antigen.

10. The method of claim 9, wherein the binding condition in step (i) is more stringent than that of step (i) in claim 7.

11. The method of claim 1, wherein step d) comprises
i) isolating mRNA from the subpopulation of cells,
ii) amplifying the mRNA into cDNA;
iii) cloning the cDNA into a cloning vector; and
iv) determining the sequence of the DNA.

12. The method of claim 1, wherein the initial mammalian display libraries are produced from any of the bone marrow, spleen, lymph nodes, and lymphocytes.

13. The method of claim 1, wherein the initial mammalian display library is produced from human.

14. The method of claim 1, wherein the antibody is a full length antibody.

15. The method of claim 14, wherein step a) comprises screening an initial population of mammalian cells transfected with first primary library of polynucleotides encoding a light chain and a second primary library encoding a heavy chain and displaying the full length antibody on the cell surface.

16. A method of isolating a polynucleotide that encodes an antibody specifically recognizing an antigen, comprising:
a) screening an initial population of mammalian cells transfected with a first primary library of polynucleotides encoding a light chain and second primary library of polynucleotides encoding a heavy chain for cells displaying an antibody specifically recognizing the antigen and recovering a subpopulation of mammalian cells;
b) reverse transcribing mRNA extracted from said subpopulation of mammalian cells into cDNA and amplifying said cDNA to generate a first sublibrary of polynucleotides encoding the light chain and a second sublibrary of polynucleotides encoding the heavy chain;
c) screening a population of mammalian cells transfected with the first sublibrary of polynucleotides and the second sublibrary of the polynucleotides for cells displaying an antibody specifically recognizing the antigen and recovering a second subpopulation of mammalian cells;
d) reverse transcribing mRNA extracted from said second subpopulation of mammalian cells into cDNA and amplifying said cDNA to generate a third sublibrary of polynucleotides encoding the light chain and the heavy chain;
e) screening a population of mammalian cells transfected with the third sublibrary of polynucleotides for cells displaying an antibody specifically recognizing the antigen and recovering a third subpopulation of mammalian cells; and
f) isolating from said third subpopulation of mammalian cells a polynucleotide encoding an antibody specifically recognizing the antigen.

17. A method of constructing a library of polynucleotides encoding antibodies, wherein the polynucleotides collectively encode at least about 10⁹ different recombinant antibodies.

18. A population of mammalian cells transiently transfected with polynucleotides encoding antibodies, wherein the polynucleotides collectively encode at least about 10⁹ different recombinant antibodies.

19. An expression vector comprising an open reading frame flanked by a pair of cleavage sites recognizable by a restriction enzyme, wherein the ends of each fragment resulting from the cleavage with said restriction enzyme do not self-ligate.

20. A dual-expression cassette vector, comprising: 1) a first open reading frame flanked by first pair of cleavage sites recognizable by a restriction enzyme, wherein the ends of each fragment resulting from the cleavage with said restriction enzyme do not self-ligate; and 2) a second open reading frame flanked by a second pair of cleavage sites recognizable by a restriction enzyme, wherein the ends of each fragment resulting from the cleavage with said second restriction enzyme do not self-ligate.
